(19) United States
(12) Patent Application Publication
(10) Pub. No.: US 2026/0001926 A1
(43) Pub. Date: Jan. 1, 2026

(54) ENGINEERED FIBROBLAST GROWTH FACTOR VARIANTS

(71) Applicant: MULTUS BIOTECHNOLOGY LIMITED, London (GB)

(72) Inventors: Kevin PAN, London (GB); George TAYLOR, London (GB); Seong Hyun (Brandon) MA, London (GB)

(21) Appl. No.: 18/845,606

(22) PCT Filed: Mar. 10, 2023
(86) PCT No.: PCT/EP2023/056205

§ 371 Date: Sep. 10, 2024

(30) Foreign Application Priority Data
Mar. 11, 2022 (GB) 2203412.8

(51) Int. Cl. C07K 14/50; C12N 5/071

(52) U.S. Cl. CPC C07K 14/50; C12N 5/0686; C12N 2501/115

(57) ABSTRACT

The invention relates to improved growth factor polypeptide variants, in particular polypeptides comprising a sequence of any one of SEQ ID NOs: 2-7 or a sequence with at least 80% similarity thereto, wherein the FGF polypeptide exhibits increased bioactivity, solubility and/or stability compared to the wild-type FGF2 polypeptide of SEQ ID NO: 1. The invention further provides nucleic acid sequences encoding an FGF polypeptide of the invention and a method of growing an animal cell comprising cultivating the animal cell in a culture medium comprising an FGF polypeptide of the invention is also provided.

### CROSS-REFERENCE TO RELATED APPLICATIONS

This patent application is a U.S. national stage application under 35 U.S.C. § 371 of International Patent Application No. PCT/EP2023/056205 filed on 10 Mar. 2023, which claims foreign priority to GB Application No. 2203412.8 filed on 11 Mar. 2022. The entire disclosures of each of the above recited applications are incorporated herein by reference.

### REFERENCE TO AN ELECTRONIC SEQUENCE LISTING

This application contains references to nucleic acid sequences and/or amino acid sequences which have been submitted concurrently herewith as the sequence listing XML file entitled “ST26_SL_10_Sept_2024.xml”, file size 15,816 Bytes (B), created on 10 Sep. 2024. The aforementioned sequence listing is hereby incorporated by reference in its entirety.

### FIELD OF THE INVENTION

The invention relates to improved growth factor polypeptide variants, in particular variants of fibroblast growth factors (FGF) and uses thereof, including use in growth media.

### BACKGROUND OF THE INVENTION

Growth factors are naturally occurring cell signalling molecules that play a number of essential roles including regulating cell proliferation and development and cellular differentiation.

One of the most common mechanisms for controlling cell signals in organisms is decay of the signalling molecule. Hence, most wild-type growth factors have high turnover rates and are not “built” to last, making them less suitable for the industrial setting where a constant level of stimulus for cell proliferation should be maintained.

There are a number of disadvantages related to this rapid degradation of wild-type growth factors, including the wasteful and inefficient use of components and introduction of variability in the context of cell culture.

Growth medium used in cell culture usually includes combinations of growth factors. To overcome the exponential depletion of these naturally occurring cell growth signalling stimuli, high levels of growth factors are used to keep the stimulus above a minimal threshold and the media must be replaced frequently (for example, every two days) even where there are still enough nutrients.

In the growing field of cultivated meat, cell culture is a fundamental aspect of the process. One of the limiting steps in the production of cultivated meat is the high cost of cell growth media. Climate and other environmental concerns are continuing to drive the demand for cultivated meat, and therefore also the need for improved growth media to replace animal serum-based media.

To address these issues, a series of engineered polypeptides are provided with improved properties, such as increased bioactivity, solubility and/or stability (for example, increased thermostability). These polypeptides can be used as a replacement for naturally occurring wild-type growth factors in growth media. The improved properties also make the polypeptides useful in a number of other areas.

Fibroblast growth factors (FGF) are a family of cell signalling proteins, and members of this family are involved in a wide variety of cellular processes. FGF polypeptides are commonly used in growth media and there is an ongoing need for FGF polypeptides with improved properties, such as increased bioactivity, solubility and/or stability (for example, increased thermostability). FGF2 is one member of this family and binds to specific FGF receptors. FGF2 can stimulate proliferation and differentiation of various cell types, and aid in the process of angiogenesis.

The present invention meets this need by providing novel polypeptides with improved properties.

### SUMMARY OF THE INVENTION

Provided herein is a fibroblast growth factor (FGF) polypeptide comprising at least one modification selected from the group consisting of an amino acid substitution, an amino acid deletion, an amino acid insertion, and combinations thereof relative to the sequence set forth in SEQ ID NO: 1, wherein the modified FGF polypeptide exhibits increased bioactivity, solubility and/or stability (for example, increased thermostability) compared to the FGF2 polypeptide of SEQ ID NO: 1.

Also provided herein is an FGF polypeptide comprising at least one modification selected from the group consisting of an amino acid substitution, an amino acid deletion, an amino acid insertion, and combinations thereof relative to the sequence set forth in SEQ ID NO: 1, wherein the modified FGF polypeptide exhibits increased bioactivity compared to the FGF2 polypeptide of SEQ ID NO: 1.

Also provided herein is an FGF polypeptide comprising a sequence of any one of SEQ ID NOs: 2-7 or a sequence with at least 80% similarity thereto, wherein the FGF polypeptide exhibits increased bioactivity, solubility and/or stability (for example, increased thermostability) compared to the wild-type FGF2 polypeptide of SEQ ID NO: 1.

Also provided herein is an FGF polypeptide comprising a sequence of SEQ ID NO: 4 or SEQ ID NO: 7 or a sequence with at least 80% similarity thereto, wherein the FGF polypeptide exhibits increased bioactivity, solubility and/or stability (for example, increased thermostability) compared to the wild-type FGF2 polypeptide of SEQ ID NO: 1.

Also provided herein is an FGF polypeptide comprising a sequence of SEQ ID NO: 4 or SEQ ID NO: 7 or a sequence with at least 80% similarity thereto, wherein the FGF polypeptide exhibits increased bioactivity compared to the wild-type FGF2 polypeptide of SEQ ID NO: 1.

Also provided herein is a nucleic acid sequence encoding an FGF polypeptide of the invention.

Further provided is a cell comprising a polypeptide of the invention or a nucleic acid sequence of the invention.

A method of growing an animal cell comprising cultivating the animal cell in a culture medium comprising an FGF polypeptide of the invention is also provided.

### BRIEF DESCRIPTION OF THE FIGURES

FIG. 1 shows alignment of the amino acid sequence of polypeptides of the invention (SEQ ID NOs: 2-7).

FIG. 2 shows alignment of the amino acid sequence of polypeptides of the invention (SEQ ID NOs: 2-7) and the wild-type FGF2 sequence (SED ID NO: 1).

FIGS. 3A-3F illustrate schematic views of the plasmid insertions encoding the polypeptides of SEQ ID NOS: 2-7.

FIGS. 4A and 4B illustrate the results of sodium dodecyl sulfate-polyacrylamide gel electrophoresis (SDS-PAGE) and western blot analyses, which show expression of the polypeptides of the invention.

FIGS. 5A-5F show a comparison of receptor kinase activity (bioactivity) of FGF polypeptides of the invention compared to wild-type FGF2. The black lines represent polypeptides of the invention and the grey line represents wild-type FGF2.

FIGS. 6A and 6B show a comparison of bioactivity of two of the FGF polypeptides of the invention compared to wild-type FGF2 measured using a ONE-Glo luciferase assay. The black lines represent polypeptides of the invention and the grey line represents wild-type FGF2.

### DETAILED DESCRIPTION OF THE INVENTION

The present invention provides novel FGF polypeptide sequences, including FGF2 polypeptide sequences, with improved properties over known FGF polypeptides, such as increased bioactivity, solubility and/or stability (for example, thermostability (melting temperature Tm)). The present invention also provides nucleic acids, encoding the FGF polypeptides of the present invention.

Unless defined otherwise, all technical and scientific terms used herein generally have the same meaning as commonly understood by a person skilled in the art to which this invention belongs. Generally, the nomenclature used herein and the laboratory procedures in cell culture, molecular genetics, organic chemistry, nucleic acid chemistry and hybridisation are those well-known and commonly employed in the art. Standard techniques are used for nucleic acid and peptide synthesis. The techniques and procedures are generally performed according to conventional methods in the art. The nomenclature used herein, and the laboratory procedures of synthetic biology described below, are those well-known and commonly employed in the art.

### Polypeptides of the Invention

In some embodiments, the invention provides a polypeptide comprising at least one modification selected from the group consisting of an amino acid substitution, an amino acid deletion, an amino acid insertion, and combinations thereof relative to the sequence set forth in SEQ ID NO: 1, wherein the modified polypeptide exhibits increased thermostability compared to the FGF2 polypeptide of SEQ ID NO: 1.

In some embodiments, the invention provides a polypeptide comprising at least one modification selected from the group consisting of an amino acid substitution, an amino acid deletion, an amino acid insertion, and combinations thereof relative to the sequence set forth in SEQ ID NO: 1, wherein the modified polypeptide is bioactive, and exhibits changes to stability and/or solubility, compared to the FGF2 polypeptide of SEQ ID NO: 1.

In some embodiments, the invention provides a polypeptide comprising at least one modification selected from the group consisting of an amino acid substitution, an amino acid deletion, an amino acid insertion, and combinations thereof relative to the sequence set forth in SEQ ID NO: 1, wherein the modified polypeptide is bioactive and exhibits stability and/or solubility at a level comparable to that exhibited by the FGF2 polypeptide of SEQ ID NO: 1.

In some embodiments, the invention provides a polypeptide comprising at least one modification selected from the group consisting of an amino acid substitution, an amino acid deletion, an amino acid insertion, and combinations thereof relative to the sequence set forth in SEQ ID NO: 1, wherein the modified polypeptide is bioactive and exhibits increased stability and/or solubility relative to that exhibited by the FGF2 polypeptide of SEQ ID NO: 1.

In some embodiments, the polypeptide of the invention is an FGF polypeptide.

In some embodiments, the polypeptide of the invention is an FGF2 polypeptide.

In some embodiments, the polypeptide of the invention binds at least one fibroblast growth factor receptor (FGFR).

In some embodiments, the polypeptide of the invention binds at least FGFR2.

In some embodiments, the polypeptide of the present invention is a recombinant polypeptide, a natural polypeptide or a synthetic polypeptide, preferably a recombinant polypeptide.

The substitution may be a conservative amino acid substitution, meaning a substitution of one amino acid residue for another sharing chemical and physical properties of the amino acid side chain (e.g., charge, size, hydrophobicity/hydrophilicity). Conservative substitutions are intended to include substitution within the following groups of amino acid residues: gly, ala; val, ile, leu; asp, glu; asn, gin; ser, thr; lys, arg; and phe, tyr.

The terms ‘wild-type FGF2’, ‘WT FGF2’, ‘naturally occurring FGF2’ or ‘natural FGF2’ refer to FGF2 that occurs in nature, without modification.

Bovine wild-type FGF2 protein comprises the sequence of SEQ ID NO: 1, and is shown below:
ALPEDGGSGAFPPGHFKDPKRLYCKNGGFFLRIHPDGRVDGVREKSDPHIKLQLQAEERGVVS IKGVCANRYLAMKEDGRLLASKCVTDECFFFERLESNNYNTYRSRKYSSWYVALKRTGQYKLG PKTGPGQKAILFLPMSAKS

FGF2 has a number of functions, such as stimulating proliferation and differentiation of various cell types, and can aid in the angiogenesis process.

FGF2 binds to fibroblast growth factor receptors (FGFRs), stimulating the receptors. This bioactivity is required for effective function of FGF2 proteins, both for naturally occurring and engineered varieties of FGF2.

In some embodiments, the polypeptide of the invention exhibits increased stability (for example, increased thermostability) compared to the FGF2 polypeptide of SEQ ID NO: 1. In some embodiments, the polypeptide of the invention exhibits increased bioactivity compared to the FGF2 polypeptide of SEQ ID NO: 1. In some embodiments, the polypeptide of the invention exhibits increased solubility, for example in Escherichia coli (E. coli) or yeast, compared to the FGF2 polypeptide of SEQ ID NO: 1. In some embodiments, the polypeptide of the invention exhibits both increased stability (for example, increased thermostability) and increased bioactivity compared to the FGF2 polypeptide of SEQ ID NO: 1. In some embodiments, the polypeptide of the invention exhibits increased stability (for example, increased thermostability), increased bioactivity and increased solubility compared to the FGF2 polypeptide of SEQ ID NO: 1.

Stability can be measured, for example, by thermostability and/or bioactivity over time.

A preferred approach for measuring stability, including thermostability, is by assessing bioactivity over time, such as by using the ONE-Glo® Luciferase assay (Promega). ONE-Glo® luciferase assay detects and quantifies expression of a luciferase reporter gene, in response to a signal transduction triggered by FGF binding to FGFR.

The assay for measuring bioactivity over time can comprise incubating polypeptides of the invention at between 30° C. and 40° C., including at about 37° C., for about 8 to 15 days, including for 10 days, with mammalian cells transfected with a luciferase reporter gene. Samples are collected every 24 hours. Bioactivity of each sample is measured by detecting expression of the luciferase gene. An example of this type of assay is the ONE-Glo® Luciferase assay (Promega) described in Example 4. Bioactivity of each protein sample was determined by producing an EC50 derived from a sigmoidal curve and comparing to the wild-type protein (the FGF2 polypeptide of SEQ ID NO: 1) incubated under identical conditions.

In some embodiments, a polypeptide of the invention has a lower EC50 than the FGF2 polypeptide of SEQ ID NO: 1 for at least 10 days, when the EC50 of each polypeptide is measured under the same conditions.

It will be understood that in order to compare the EC50 of a polypeptide of the invention to the EC50 of the FGF2 polypeptide of SEQ ID NO: 1, the EC50 of both are measured in the same experiment and under the same conditions. Therefore, in an embodiment, improved bioactivity over time of a polypeptide of the invention means a lower EC50 than the FGF2 polypeptide of SEQ ID NO: 1, when measured under the same conditions for at least 10 days.

In some embodiments, the polypeptide of the invention has an EC50 at least 2-fold lower than the EC50 of the wild-type FGF2 polypeptide of SEQ ID NO: 1 for at least 10 days. In some embodiments, the polypeptide of the invention has an EC50 at least 5-fold lower than the EC50 of the wild-type FGF2 polypeptide of SEQ ID NO: 1 for at least 10 days. In some embodiments, the polypeptide of the invention has an EC50 at least 10-fold lower than the EC50 of the wild-type FGF2 polypeptide of SEQ ID NO: 1 for at least 10 days.

In some embodiments, the polypeptide of the invention has an EC50 that is substantially the same or lower than the EC50 of the wild-type FGF2 polypeptide of SEQ ID NO: 1 for at least 10 days.

The term “thermostability” refers to the resistance of a polypeptide to irreversible changes in protein structure when heated. Wild-type FGF2 polypeptide has a melting temperature (Tm) of about 55° C. Thermostability can be measured by Differential Scanning Fluorometry, where changes in fluorescence of a dye that binds preferentially to unfolded protein over folded protein, for example Spyro Orange, are measured by PCR, for example, real time PCR, and a melting temperature (° C.) is determined. This assay is described in detail in Example 7. Melting temperature quantifies thermostability.

In some embodiments, the polypeptide of the invention exhibits a melting temperature substantially the same as the melting temperature of the FGF2 polypeptide of SEQ ID NO: 1. In this context, substantially the same is within 5° C. of the melting temperature of the FGF2 polypeptide of SEQ ID NO: 1.

In some embodiments, the polypeptide of the invention exhibits a melting temperature higher than the melting temperature of the FGF2 polypeptide of SEQ ID NO: 1. The increase in melting temperature may be around 1° C., 2° C., 3° C., 4° C., 5° C., 10° C., 15° C., 20° C., 25° C. or 30° C.

In some embodiments, the polypeptide of the invention has a melting temperature at least 2° C. higher than the melting temperature of the FGF2 polypeptide of SEQ ID NO: 1.

In some embodiments, the polypeptide of the invention exhibits an increase in melting temperature of between 1° C. and 30° C. compared to the FGF2 polypeptide of SEQ ID NO: 1.

In some embodiments, the polypeptide of the invention exhibits an increase in melting temperature of between 5° C. and 30° C. compared to the FGF2 polypeptide of SEQ ID NO: 1.

In some embodiments, the polypeptide of the invention exhibits an increase in melting temperature of between 10° C. and 30° C. compared to the FGF2 polypeptide of SEQ ID NO: 1.

In some embodiments, the polypeptide of the invention exhibits an increase in melting temperature of between 15° C. and 30° C. compared to the FGF2 polypeptide of SEQ ID NO: 1.

In some embodiments, the polypeptide of the invention exhibits an increase in melting temperature of between 20° C. and 30° C. compared to the FGF2 polypeptide of SEQ ID NO: 1.

In some embodiments, the polypeptide of the invention exhibits an increase in melting temperature of between 25° C. and 30° C. compared to the FGF2 polypeptide of SEQ ID NO: 1.

In some embodiments, the polypeptide of the invention exhibits an increase in melting temperature of between 10° C. and 25° C. compared to the FGF2 polypeptide of SEQ ID NO: 1.

In some embodiments, the polypeptide of the invention exhibits an increase in melting temperature of between 15° C. and 20° C. compared to the FGF2 polypeptide of SEQ ID NO: 1.

In some embodiments, the polypeptide of the invention exhibits an increase in melting temperature of at least 5° C. In some embodiments, the polypeptide of the invention exhibits an increase in melting temperature of at least 10° C. In some embodiments, the polypeptide of the invention has a melting temperature of at least 55° C. In some embodiments, the polypeptide of the invention has a melting temperature of at least 60° C. In some embodiments, the polypeptide of the invention has a melting temperature of at least 65° C. In some embodiments, the polypeptide of the invention has a melting temperature of at least 70° C. In some embodiments, the polypeptide of the invention has a melting temperature of at least 75° C.

The term “bioactive” refers to the ability of a polypeptide to bind a relevant receptor. Bioactivity as used herein can refer to the ability of a polypeptide of the invention to bind a fibroblast growth factor receptor (FGFR), such as FGFR2. Receptor binding can be determined by a luminescent ADP detection system, where ADP formed from a kinase reaction is measured. This assay is described in detail in Example 4. EC50 (half maximal effective concentration) quantifies bioactivity.

Bioactivity can be determined by incubating polypeptides of the invention at between 30° C. and 40° C., including at about 37° C. with mammalian cells transfected with a luciferase reporter gene. Samples were collected and bioactivity of each sample is measured by detecting expression of the luciferase gene. An example of this type of assay is the ONE-Glo® Luciferase assay (Promega) described in Example 4. Bioactivity of each protein sample was compared by producing an EC50 derived from a sigmoidal curve to the wild-type protein incubated under identical conditions.

In some embodiments, a polypeptide of the invention has a lower EC50 than the FGF2 polypeptide of SEQ ID NO: 1 when the EC50 of each polypeptide is measured under the same conditions.

It will be understood that in order to compare the EC50 of a polypeptide of the invention to the EC50 of the FGF2 polypeptide of SEQ ID NO: 1, the EC50 of both are measured in the same experiment and under the same conditions. Therefore, in an embodiment, improved bioactivity of a polypeptide of the invention means a lower EC50 than the FGF2 polypeptide of SEQ ID NO: 1, when measured under the same conditions.

In some embodiments, the polypeptide of the invention has an EC50 at least 2-fold lower than the EC50 of the wild-type FGF2 polypeptide of SEQ ID NO: 1. In some embodiments, the polypeptide of the invention has an EC50 at least 5-fold lower than the EC50 of the wild-type FGF2 polypeptide of SEQ ID NO: 1. In some embodiments, the polypeptide of the invention has an EC50 at least 10-fold lower than the EC50 of the wild-type FGF2 polypeptide of SEQ ID NO: 1.

In some embodiments, the polypeptide of the invention has an EC50 that is substantially the same or lower than the EC50 of the wild-type FGF2 polypeptide of SEQ ID NO: 1.

Wild-type FGF2 polypeptide has an EC50 of approximately 0.2 ng/ml when determined in HEK293T cells transfected with a plasmid as deposited under GenBank® accession number FJ773212. In some embodiments, a polypeptide of the invention exhibits an EC50 of below 0.2 ng/ml when determined in HEK293T cells transfected with a plasmid as deposited under GenBank® accession number FJ773212.

The plasmid deposited as FJ773212 is produced by Promega as pGL4.33.

In some embodiments, the polypeptide of the invention has an EC50 of less than 0.2 ng/ml and equal to or above 0.05 ng/ml, including between 0.05 ng/ml and 0.15 ng/ml when determined in HEK293T cells transfected with a plasmid as deposited under GenBank® accession number FJ773212. In some embodiments, a polypeptide of the invention exhibits an EC50 of less than 0.2 ng/ml and equal to or above 0.05 ng/ml when determined using a kinase assay, such as that described in Example 3.

In some embodiments, the polypeptide of the invention has an EC50 at least 0.05 ng/ml below the EC50 of the wild-type FGF2 polypeptide of SEQ ID NO: 1. In some embodiments, the polypeptide of the invention has an EC50 at least 0.075 ng/ml below that of the wild-type FGF2 polypeptide of SEQ ID NO: 1. In some embodiments, the polypeptide of the invention has an EC50 at least 0.1 ng/ml below that of the wild-type FGF2 polypeptide of SEQ ID NO: 1. In some embodiments, the polypeptide of the invention has an EC50 at least 0.125 ng/ml below that of the wild-type FGF2 polypeptide of SEQ ID NO: 1. In some embodiments, the polypeptide of the invention has an EC50 at least 0.15 ng/ml below that of the wild-type FGF2 polypeptide of SEQ ID NO: 1.

In some embodiments, the polypeptide of the invention is capable of binding a fibroblast growth factor receptor (FGFR). In some embodiments, the polypeptide of the invention is capable of binding to FGFR1, FGFR2, FGFR3 and/or FGFR4. In some embodiments, the polypeptide of the invention is capable of binding to FGFR1, FGFR2, FGFR3 and FGFR4.

“Solubility” as used herein refers to ability of the polypeptide to fold into a functional protein. Solubility can be an indication of expression of functional protein by a particular expression platform. Solubility can be determined by using SDS-PAGE and western blot. This assay is described in detail in Example 5. Soluble fraction (%) quantifies solubility.

In some embodiments, the polypeptide of the invention has a solubility of at least 30%. In some embodiments, the polypeptide of the invention has a solubility of at least 40%. In some embodiments, the polypeptide of the invention has a solubility of at least 50%. In some embodiments, the polypeptide of the invention has a solubility of at least 60%. In some embodiments, the polypeptide of the invention has a solubility of at least 70%. In some embodiments, the polypeptide of the invention has a solubility of at least 80%. In some embodiments, the polypeptide of the invention has a solubility of at least 90%. In some embodiments, the polypeptide of the invention has a solubility of 100%.

In some embodiments, the polypeptide of the invention is more stable than wild-type FGF2 (e.g. a polypeptide of SEQ ID NO: 1) and has higher or substantially the same bioactivity levels. In some embodiments, the polypeptide of the invention is more stable than wild-type FGF2 (e.g. a polypeptide of SEQ ID NO: 1) and exhibits substantially the same solubility.

A polypeptide of the invention with substantially the same solubility as wild-type FGF2 has solubility within 5% of wildtype FGF2, including within 4%, 3%, 2% or 1%.

In some embodiments, the polypeptide of the invention is more thermostable than wild-type FGF2 (e.g. a polypeptide of SEQ ID NO: 1) and has comparable bioactivity levels. In some embodiments, the polypeptide of the invention is more thermostable than wild-type FGF2 (e.g. a polypeptide of SEQ ID NO: 1) and exhibits comparable solubility.

In some embodiments, the polypeptide of the invention is soluble and bioactive. In some embodiments, the polypeptide of the invention is bioactive, soluble and thermostable at 40° C., 50° C. or 60° C.

In some embodiments, the polypeptide of the invention is soluble and exhibits substantially the same stability as compared to the FGF2 polypeptide of SEQ ID NO: 1, or is more stable. In some embodiments, the polypeptide of the invention is bioactive and exhibits increased stability compared to the FGF2 polypeptide of SEQ ID NO: 1. In some embodiments, the polypeptide of the invention is soluble and bioactive and exhibits increased stability compared to the FGF2 polypeptide of SEQ ID NO: 1.

In some embodiments, the polypeptide of the invention is soluble and exhibits increased thermostability compared to the FGF2 polypeptide of SEQ ID NO: 1. In some embodiments, the polypeptide of the invention is bioactive and exhibits increased thermostability compared to the FGF2 polypeptide of SEQ ID NO: 1. In some embodiments, the polypeptide of the invention is bioactive and soluble and exhibits increased thermostability compared to the FGF2 polypeptide of SEQ ID NO: 1.

In some embodiments, the polypeptide of the invention comprises a modification at position 34 of SEQ ID NO: 1. In some embodiments, the polypeptide of the invention comprises a substitution of histidine at position 34 of SEQ ID NO: 1. In some embodiments, the polypeptide of the invention comprises an H34E substitution at position 34 of SEQ ID NO: 1.

In some embodiments, the polypeptide of the invention comprises a modification at position 89 of SEQ ID NO: 1. In some embodiments, the polypeptide of the invention comprises a substitution of aspartic acid at position 89 of SEQ ID NO: 1. In some embodiments, the polypeptide of the invention comprises an D89E substitution at position 89 of SEQ ID NO: 1.

In some embodiments, the polypeptide of the invention comprises a substitution of histidine at position 34 of SEQ ID NO: 1 and a substitution of aspartic acid at position 89 of SEQ ID NO: 1. In some embodiments, the polypeptide of the invention comprises an H34E substitution at position 34 of SEQ ID NO: 1 and an D89E substitution at position 89 of SEQ ID NO: 1.

In some embodiments, the polypeptide of the invention comprises the amino acid sequence

GFFLRIEXaa38Xaa39Xaa40Xaa41Xaa42Xaa43Xaa44Xaa45
Xaa46Xaa47Xaa48Xaa49Xaa50Xaa51Xaa52Xaa53Xaa54Xaa
55Xaa56KLQLQAEERXaa66GVVXaa70IKGVXaa75Xaa76Xaa77
Xaa78YLAMKEDGRLXaa89Xaa90Xaa91Xaa92Xaa93Xaa94Xaa
95Xaa96EEC,

wherein:
Xaa38 is P, D or a deletion, Xaa39 is P or a deletion, Xaa40 is D or G, Xaa41 is G or a deletion, Xaa42 is R or a deletion, Xaa43 is V or a deletion, Xaa44 is D, E or K, Xaa45 is G, D or V, Xaa46 is V, A or D, Xaa47 is G or a deletion, Xaa48 is R or V, Xaa49 is E or R, Xaa50 is K or E, Xaa51 is K, S or a deletion, Xaa52 is Q, S, D or a deletion, Xaa53 is D or P, Xaa54 is P, D or a deletion, Xaa55 is H or a deletion, Xaa56 is I or a deletion, Xaa66 is G or a deletion, Xaa70 is L, S or T, Xaa75 is L, P or a deletion, Xaa76 is A, D or a deletion, Xaa77 is N, L or I, Xaa78 is R, K or a deletion, Xaa89 is L or F, Xaa90 is Y, A or a deletion, Xaa91 is A, L or E, Xaa92 is L, S or a deletion, Xaa93 is E, K, P, A or a deletion, Xaa94 is C, L, E or a deletion, Xaa95 is P or A, Xaa96 is E, T or a deletion.

In some embodiments, the polypeptide of the invention comprises a sequence of any one of SEQ ID NOs: 2-7.

In some embodiments, the polypeptide of the invention consists of the sequence of any one of SEQ ID NOs: 2-7.

In some embodiments, the polypeptide of the invention comprises a sequence with at least 80% similarity to any one of SEQ ID NOs: 2-7. In some embodiments, the polypeptide of the invention comprises a sequence with at least 85% similarity to any one of SEQ ID NOs: 2-7. In some embodiments, the polypeptide of the invention comprises a sequence with at least 90% similarity to any one of SEQ ID NOs: 2-7. In some embodiments, the polypeptide of the invention comprises a sequence with at least 95% similarity to any one of SEQ ID NOs: 2-7. The polypeptide of the embodiment can be an FGF2 polypeptide. The polypeptide of this embodiment can bind at least one fibroblast growth factor receptor (FGFR).

In a preferred embodiment, the polypeptide of the invention comprises a sequence with at least 95% similarity to any one of SEQ ID NOs: 2-7.

In some embodiments, the polypeptide of the invention comprises a sequence of SEQ ID NO: 4 or SEQ ID NO: 7.

In some embodiments, the polypeptide of the invention comprises a sequence of SEQ ID NO: 4.

In some embodiments, the polypeptide of the invention comprises a sequence of SEQ ID NO: 7.

In some embodiments, the polypeptide of the invention consists of the sequence of SEQ ID NO: 4 or SEQ ID NO: 7.

In some embodiments, the polypeptide of the invention consists of the sequence of SEQ ID NO: 4.

In some embodiments, the polypeptide of the invention consists of the sequence of SEQ ID NO: 7.

In some embodiments, the polypeptide of the invention comprises a sequence with at least 80% similarity to SEQ ID NO: 4 or SEQ ID NO: 7. In some embodiments, the polypeptide of the invention comprises a sequence with at least 85% similarity to SEQ ID NO: 4 or SEQ ID NO: 7. In some embodiments, the polypeptide of the invention comprises a sequence with at least 90% similarity to SEQ ID NO: 4 or SEQ ID NO: 7. In some embodiments, the polypeptide of the invention comprises a sequence with at least 95% similarity to SEQ ID NO: 4 or SEQ ID NO: 7. The polypeptide of the embodiment can be an FGF2 polypeptide. The polypeptide of this embodiment can bind at least one fibroblast growth factor receptor (FGFR).

In some embodiments, the polypeptide of the invention comprises a sequence with at least 80% similarity to SEQ ID NO: 4. In some embodiments, the polypeptide of the invention comprises a sequence with at least 85% similarity to SEQ ID NO: 4. In some embodiments, the polypeptide of the invention comprises a sequence with at least 90% similarity to SEQ ID NO: 4. In some embodiments, the polypeptide of the invention comprises a sequence with at least 95%, including 98% and 99%, similarity to SEQ ID NO: 4. The polypeptide of the embodiment can be an FGF2 polypeptide. The polypeptide of this embodiment can bind at least one fibroblast growth factor receptor (FGFR).

In some embodiments, the polypeptide of the invention comprises a sequence with at least 80% similarity to SEQ ID NO: 7. In some embodiments, the polypeptide of the invention comprises a sequence with at least 85% similarity to SEQ ID NO: 7. In some embodiments, the polypeptide of the invention comprises a sequence with at least 90% similarity to SEQ ID NO: 7. In some embodiments, the polypeptide of the invention comprises a sequence with at least 95%, including 98% and 99%, similarity to SEQ ID NO: 7. The polypeptide of the embodiment can be an FGF2 polypeptide. The polypeptide of this embodiment can bind at least one fibroblast growth factor receptor (FGFR).

In a preferred embodiment, the polypeptide of the invention comprises a sequence with at least 95% similarity to SEQ ID NO: 4 or SEQ ID NO: 7 that can bind at least one fibroblast growth factor receptor (FGFR).

In a preferred embodiment, the polypeptide of the invention comprises a sequence with at least 95% similarity to SEQ ID NO: 4 that can bind at least one fibroblast growth factor receptor (FGFR).

In a preferred embodiment, the polypeptide of the invention comprises a sequence with at least 95% similarity to SEQ ID NO: 7 that can bind at least one fibroblast growth factor receptor (FGFR).

Percent similarity (or ‘percentage similarity’) between two sequences can be calculated by multiplying the number of matches in the pair by 100 and dividing by the length of the aligned region, including gaps. Identity scoring only counts perfect matches and does not consider the degree of similarity of amino acids to one another. Gaps at the end of sequences are not included, and internal gaps are included in the length.

Alignments can be generated using programs known in the art. For purposes herein, alignment of nucleotide sequences can be performed with the blastn program set at default parameters, and alignment of amino acid sequences can be performed with the blastp program set at default parameters (see National Center for Biotechnology Information (NCBI): ncbi.nlm.nih.gov).

In some embodiments, the polypeptide of the invention comprises the sequence of SEQ ID NO: 6 or SEQ ID NO: 7. In some embodiments, the polypeptide of the invention comprises a sequence with at least 80% similarity to the sequence of SEQ ID NO: 6 or SEQ ID NO: 7. In some embodiments, the polypeptide of the invention comprises a sequence with at least 85% similarity to the sequence of SEQ ID NO: 6 or SEQ ID NO: 7. In some embodiments, the polypeptide of the invention comprises a sequence with at least 90% similarity to the sequence of SEQ ID NO: 6 or SEQ ID NO: 7. In some embodiments, the polypeptide of the invention comprises a sequence with at least 95% similarity to the sequence of SEQ ID NO: 6 or SEQ ID NO: 7. In some embodiments, the polypeptide of the invention consists of the sequence of SEQ ID NO: 6 or SEQ ID NO: 7.

In some embodiments, the polypeptide of the invention comprises the sequence of any one of SEQ ID NOs: 2-5. In some embodiments, the polypeptide of the invention comprises a sequence with at least 80% similarity to any one of SEQ ID NOs: 2-5. In some embodiments, the polypeptide of the invention comprises a sequence with at least 85% similarity to any one of SEQ ID NOs: 2-5. In some embodiments, the polypeptide of the invention comprises a sequence with at least 90% similarity to any one of SEQ ID NOs: 2-5. In some embodiments, the polypeptide of the invention comprises a sequence with at least 95% similarity to any one of SEQ ID NOs: 2-5. In some embodiments, the polypeptide of the invention consists of the sequence of any of any one of SEQ ID NOs: 2-5.

In some embodiments, the polypeptide of the invention comprises the sequence of SEQ ID NO: 2 or SEQ ID NO: 3. In some embodiments, the polypeptide of the invention comprises a sequence with at least 80% similarity to the sequence of SEQ ID NO: 2 or SEQ ID NO: 3. In some embodiments, the polypeptide of the invention comprises a sequence with at least 85% similarity to the sequence of SEQ ID NO: 2 or SEQ ID NO: 3. In some embodiments, the polypeptide of the invention comprises a sequence with at least 90% similarity to the sequence of SEQ ID NO: 2 or SEQ ID NO: 3. In some embodiments, the polypeptide of the invention comprises a sequence with at least 95% similarity to the sequence of SEQ ID NO: 2 or SEQ ID NO: 3. In some embodiments, the polypeptide of the invention consists of the sequence of SEQ ID NO: 2 or SEQ ID NO: 3.

In some embodiments, the polypeptide of the invention comprises the sequence of SEQ ID NO: 5 or SEQ ID NO: 6. In some embodiments, the polypeptide of the invention comprises a sequence with at least 80% similarity to the sequence of SEQ ID NO: 4 or SEQ ID NO: 5. In some embodiments, the polypeptide of the invention comprises a sequence with at least 85% similarity to the sequence of SEQ ID NO: 4 or SEQ ID NO: 5. In some embodiments, the polypeptide of the invention comprises a sequence with at least 90% similarity to the sequence of SEQ ID NO: 4 or SEQ ID NO: 5. In some embodiments, the polypeptide of the invention comprises a sequence with at least 95% similarity to the sequence of SEQ ID NO: 4 or SEQ ID NO: 5. In some embodiments, the polypeptide of the invention consists of the sequence of SEQ ID NO: 4 or SEQ ID NO: 5.

In some embodiments, the polypeptide of the invention comprises the sequence of SEQ ID NO: 4 or SEQ ID NO: 7. In some embodiments, the polypeptide of the invention comprises a sequence with at least 80% similarity to the sequence of SEQ ID NO: 4 or SEQ ID NO: 7. In some embodiments, the polypeptide of the invention comprises a sequence with at least 85% similarity to the sequence of SEQ ID NO: 4 or SEQ ID NO: 7. In some embodiments, the polypeptide of the invention comprises a sequence with at least 90% similarity to the sequence of SEQ ID NO: 4 or SEQ ID NO: 7. In some embodiments, the polypeptide of the invention comprises a sequence with at least 95% similarity to the sequence of SEQ ID NO: 4 or SEQ ID NO: 7. In some embodiments, the polypeptide of the invention consists of the sequence of SEQ ID NO: 4 or SEQ ID NO: 7.

In some embodiments, the polypeptide of the invention comprises the sequence of SEQ ID NO: 2. In some embodiments, the polypeptide of the invention comprises a sequence with at least 80% similarity to the sequence of SEQ ID NO: 2. In some embodiments, the polypeptide of the invention comprises a sequence with at least 85% similarity to the sequence of SEQ ID NO: 2. In some embodiments, the polypeptide of the invention comprises a sequence with at least 90% similarity to the sequence of SEQ ID NO: 2. In some embodiments, the polypeptide of the invention comprises a sequence with at least 95% similarity to the sequence of SEQ ID NO: 2. In this embodiment, the polypeptide of the invention can be more thermostable than wild-type FGF2 (e.g. a polypeptide of SEQ ID NO: 1). In some embodiments, the polypeptide of the invention consists of the sequence of SEQ ID NO: 2.

In a preferred embodiment, the polypeptide of the invention comprises a sequence with at least 95% similarity to the sequence of SEQ ID NO: 2.

In some embodiments, the polypeptide of the invention comprises the sequence of SEQ ID NO: 3. In some embodiments, the polypeptide of the invention comprises a sequence with at least 80% similarity to the sequence of SEQ ID NO: 3. In some embodiments, the polypeptide of the invention comprises a sequence with at least 85% similarity to the sequence of SEQ ID NO: 3. In some embodiments, the polypeptide of the invention comprises a sequence with at least 90% similarity to the sequence of SEQ ID NO: 3. In some embodiments, the polypeptide of the invention comprises a sequence with at least 95% similarity to the sequence of SEQ ID NO: 3. In this embodiment, the polypeptide of the invention can be more thermostable than wild-type FGF2 (e.g. a polypeptide of SEQ ID NO: 1). In some embodiments, the polypeptide of the invention consists of the sequence of SEQ ID NO: 3.

In a preferred embodiment, the polypeptide of the invention comprises a sequence with at least 95% similarity to the sequence of SEQ ID NO: 3.

In some embodiments, the polypeptide of the invention comprises the sequence of SEQ ID NO: 4. In some embodiments, the polypeptide of the invention comprises a sequence with at least 80% similarity to the sequence of SEQ ID NO: 4. In some embodiments, the polypeptide of the invention comprises a sequence with at least 85% similarity to the sequence of SEQ ID NO: 4. In some embodiments, the polypeptide of the invention comprises a sequence with at least 90% similarity to the sequence of SEQ ID NO: 4. In some embodiments, the polypeptide of the invention comprises a sequence with at least 95% similarity to the sequence of SEQ ID NO: 4. In this embodiment, the polypeptide of the invention can be more thermostable than wild-type FGF2 (e.g. a polypeptide of SEQ ID NO: 1). In some embodiments, the polypeptide of the invention consists of the sequence of SEQ ID NO: 4.

In a preferred embodiment, the polypeptide of the invention comprises a sequence with at least 95% similarity to the sequence of SEQ ID NO: 4.

In some embodiments, the polypeptide of the invention comprises the sequence of SEQ ID NO: 5. In some embodiments, the polypeptide of the invention comprises a sequence with at least 80% similarity to the sequence of SEQ ID NO: 5. In some embodiments, the polypeptide of the invention comprises a sequence with at least 85% similarity to the sequence of SEQ ID NO: 5. In some embodiments, the polypeptide of the invention comprises a sequence with at least 90% similarity to the sequence of SEQ ID NO: 5. In some embodiments, the polypeptide of the invention comprises a sequence with at least 95% similarity to the sequence of SEQ ID NO: 5. In this embodiment, the polypeptide of the invention can be more thermostable than wild-type FGF2 (e.g. a polypeptide of SEQ ID NO: 1). In some embodiments, the polypeptide of the invention consists of the sequence of SEQ ID NO: 5.

In a preferred embodiment, the polypeptide of the invention comprises a sequence with at least 95% similarity to the sequence of SEQ ID NO: 5.

In some embodiments, the polypeptide of the invention comprises the sequence of SEQ ID NO: 6. In some embodiments, the polypeptide of the invention comprises a sequence with at least 80% similarity to the sequence of SEQ ID NO: 6. In some embodiments, the polypeptide of the invention comprises a sequence with at least 85% similarity to the sequence of SEQ ID NO: 6. In some embodiments, the polypeptide of the invention comprises a sequence with at least 90% similarity to the sequence of SEQ ID NO: 6. In some embodiments, the polypeptide of the invention comprises a sequence with at least 95% similarity to the sequence of SEQ ID NO: 6. In this embodiment, the polypeptide of the invention can be more thermostable than wild-type FGF2 (e.g. a polypeptide of SEQ ID NO: 1). In some embodiments, the polypeptide of the invention consists of the sequence of SEQ ID NO: 6.

In a preferred embodiment, the polypeptide of the invention comprises a sequence with at least 95% similarity to the sequence of SEQ ID NO: 6.

In some embodiments, the polypeptide of the invention comprises the sequence of SEQ ID NO: 7. In some embodiments, the polypeptide of the invention comprises a sequence with at least 80% similarity to the sequence of SEQ ID NO: 7. In some embodiments, the polypeptide of the invention comprises a sequence with at least 85% similarity to the sequence of SEQ ID NO: 7. In some embodiments, the polypeptide of the invention comprises a sequence with at least 90% similarity to the sequence of SEQ ID NO: 7. In some embodiments, the polypeptide of the invention comprises a sequence with at least 95% similarity to the sequence of SEQ ID NO: 7. In this embodiment, the polypeptide of the invention can be more thermostable than wild-type FGF2 (e.g. a polypeptide of SEQ ID NO: 1). In some embodiments, the polypeptide of the invention consists of the sequence of SEQ ID NO: 7.

In a preferred embodiment, the polypeptide of the invention comprises a sequence with at least 95% similarity to the sequence of SEQ ID NO: 7.

The present invention further relates to fragments, analogs and derivatives of a polypeptide of the invention, where the “fragment,” “derivative” and “analog” retains essentially the same biological function or activity as a polypeptide as set forth in any one of SEQ ID NOs: 2 to 7. Thus, the fragment, analog or derivative can have a melting temperature of at least 55° C., an EC50 equal to or below the EC50 of wild-type FGF2 (e.g. a polypeptide of SEQ ID NO: 1) when the EC50 of the fragment, derivative or analog and the EC50 of the wild-type polypeptide is determined under the same conditions, and/or solubility of above 30%.

The polypeptides and polynucleotides of the present invention are preferably provided in an isolated form, and preferably are purified to homogeneity. “Isolated” means that the polypeptide or polynucleotide is separated from its natural environment.

In some embodiments, the polypeptide of the invention comprises an insertion between glutamic acid and aspartic acid at positions 4 and 5 of SEQ ID NO: 1. In some embodiments, the insertion is an insertion of alanine.

In some embodiments, the polypeptide of the invention comprises a substitution of serine at position 8 of SEQ ID NO: 1. In some embodiments, the substitution is an S8A substitution.

In some embodiments, the polypeptide of the invention comprises an insertion before at position 27 of SEQ ID NO: 1. In some embodiments, the insertion is an insertion of DDGAE.

In some embodiments, the polypeptide of the invention comprises an insertion between proline and aspartic acid at positions 35 and 36 of SEQ ID NO: 1. In some embodiments the insertion is an insertion of proline.

In some embodiments, the polypeptide of the invention comprises an insertion between proline and aspartic acid at positions 39 and 40 of SEQ ID NO: 1. In some embodiments, the insertion is an insertion of EV.

In some embodiments, the polypeptide of the invention comprises a substitution of alanine at position 83 of SEQ ID NO: 1. In some embodiments, the substitution is an A83Y substitution.

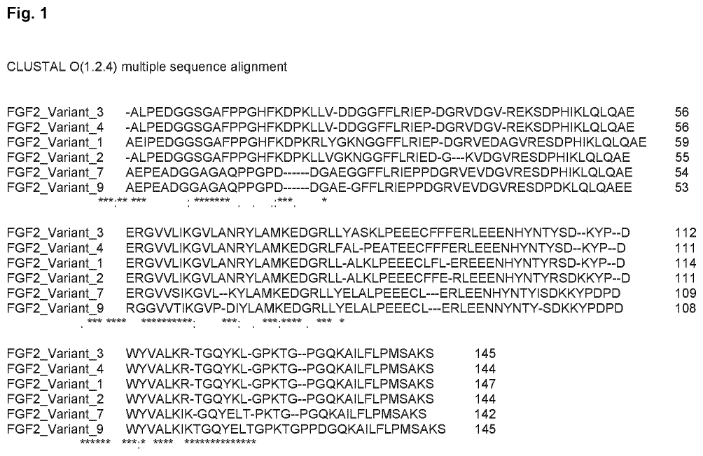

In some embodiments, the polypeptide of the invention comprises a sequence which is a consensus sequence of the alignment shown in FIG. 1.

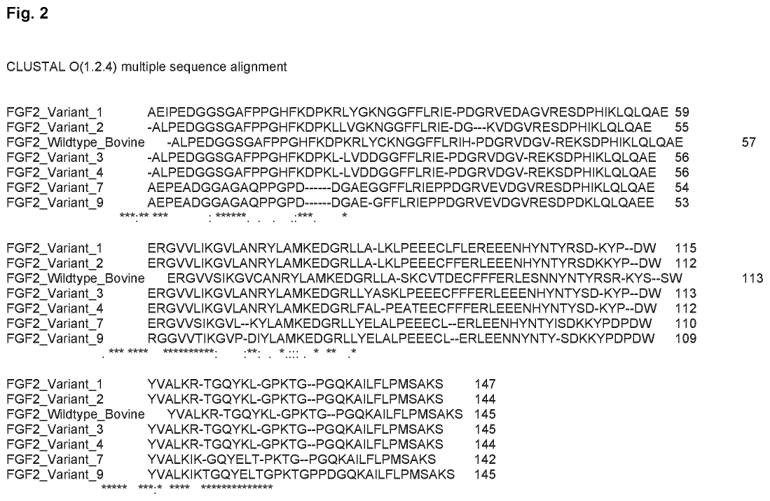

In some embodiments, the polypeptide of the invention comprises a sequence which is a consensus sequence of the alignment shown in FIG. 2.

In some embodiments, the consensus sequence comprises the amino acid residues in common between all of SEQ ID NOs: 2-7. In some embodiments, the consensus sequence comprises the amino acid residues in common between all of SEQ ID NOs: 2-7 that differ from SEQ ID NO: 1.

In some embodiments, the consensus sequence comprises the amino acid residues in common between SEQ ID NO: 4 and SEQ ID NO: 7. In some embodiments, the consensus sequence comprises the amino acid residues in common between SEQ ID NO: 4 and SEQ ID NO: 7 that differ from SEQ ID NO: 1.

In some embodiments, the polypeptide of the invention comprises a sequence which is a consensus sequence of the alignment of one of FIG. 1 or FIG. 2, wherein, for residues which are not in common between all of SEQ ID NOs: 2-7, the polypeptide of the invention comprises one of the residues of one of SEQ ID NOs: 2-7 at that position.

In some embodiments, the polypeptide of the invention comprises a sequence which is a consensus sequence of the alignment of one of FIG. 1 or FIG. 2, wherein, for residues which are not in common between SEQ ID NO: 4 and SEQ ID NO: 7, the polypeptide of the invention comprises one of the residues of one of SEQ ID NO: 4 and SEQ ID NO: 7 at that position.

In some embodiments of the invention, the minimum length of the polypeptide of the invention is 80% the length of the wild-type FGF2 sequence of SEQ ID NO: 1. In some embodiments of the invention, the minimum length of the polypeptide of the invention is 85% the length of the wild-type FGF2 sequence of SEQ ID NO: 1. In some embodiments of the invention, the minimum length of the polypeptide of the invention is 90% the length of the wild-type FGF2 sequence of SEQ ID NO: 1. In some embodiments of the invention, the minimum length of the polypeptide of the invention is 95% the length of the wild-type FGF2 sequence of SEQ ID NO: 1.

In some embodiments of the invention, the maximum length of the polypeptide of the invention is twice as long as the length of the wild-type FGF2 sequence of SEQ ID NO: 1. In some embodiments of the invention, the maximum length of the polypeptide of the invention is 80% more than the length of the wild-type FGF2 sequence of SEQ ID NO: 1. In some embodiments of the invention, the maximum length of the polypeptide of the invention is 70% more than the length of the wild-type FGF2 sequence of SEQ ID NO: 1. In some embodiments of the invention, the maximum length of the polypeptide of the invention is 60% more than the length of the wild-type FGF2 sequence of SEQ ID NO: 1. In some embodiments of the invention, the maximum length of the polypeptide of the invention is 50% more than the length of the wild-type FGF2 sequence of SEQ ID NO: 1. In some embodiments of the invention, the maximum length of the polypeptide of the invention is 40% more than the length of the wild-type FGF2 sequence of SEQ ID NO: 1. In some embodiments of the invention, the maximum length of the polypeptide of the invention is 30% more than the length of the wild-type FGF2 sequence of SEQ ID NO: 1. In some embodiments of the invention, the maximum length of the polypeptide of the invention is 20% more than the length of the wild-type FGF2 sequence of SEQ ID NO: 1. In some embodiments of the invention, the maximum length of the polypeptide of the invention is 10% more than the length of the wild-type FGF2 sequence of SEQ ID NO: 1.

In some embodiments, the polypeptide of the invention is the same length as the wild-type FGF2 sequence of SEQ ID NO: 1.

In some embodiments, the polypeptide of the invention has a length between 50% shorter and 50% longer than the wild-type FGF2 sequence of SEQ ID NO: 1. In some embodiments, the polypeptide of the invention has a length between 40% shorter and 40% longer than the wild-type FGF2 sequence of SEQ ID NO: 1. In some embodiments, the polypeptide of the invention has a length between 30% shorter and 30% longer than the wild-type FGF2 sequence of SEQ ID NO: 1. In some embodiments, the polypeptide of the invention has a length between 20% shorter and 20% longer than the wild-type FGF2 sequence of SEQ ID NO: 1. In some embodiments, the polypeptide of the invention has a length between 10% shorter and 10% longer than the wild-type FGF2 sequence of SEQ ID NO: 1. In some embodiments, the polypeptide of the invention has a length between 5% shorter and 5% longer than the wild-type FGF2 sequence of SEQ ID NO: 1. In some embodiments, the polypeptide of the invention has a length between 2% shorter and 2% longer than the wild-type FGF2 sequence of SEQ ID NO: 1.

### Nucleic Acid Sequences of the Invention

In some embodiments, the invention provides a nucleic acid sequence encoding any of the polypeptides disclosed herein.

In some embodiments, the invention provides a nucleic acid sequence encoding the polypeptide of any one of SEQ ID NOs: 2-7.

In some embodiments, the invention provides a nucleic acid sequence encoding the polypeptide of SEQ ID NO: 4 or SEQ ID NO: 7.

In some embodiments, the invention provides a nucleic acid sequence encoding the polypeptide of SEQ ID NO: 4.

In some embodiments, the invention provides a nucleic acid sequence encoding the polypeptide of SEQ ID NO: 7.

In some embodiments, the invention provides a nucleic acid sequence having at least 80% identity with the nucleic acid sequence of any one of SEQ ID NOs: 8-13. In some embodiments, the invention provides a nucleic acid sequence having at least 85% identity with the nucleic acid sequence of any one of SEQ ID NOs: 8-13. In some embodiments, the invention provides a nucleic acid sequence having at least 90% identity with the nucleic acid sequence of any one of SEQ ID NOs: 8-13. In some embodiments, the invention provides a nucleic acid sequence having at least 95% identity with the nucleic acid sequence of any one of SEQ ID NOs: 8-13. In some embodiments, the nucleic acid sequence of the invention encodes an FGF polypeptide with increased bioactivity, solubility and/or stability (for example, increased thermostability) compared to the FGF2 polypeptide of SEQ ID NO: 1.

In some embodiments, the invention provides a nucleic acid sequence having at least 80% identity with the nucleic acid sequence of SEQ ID NO: 10 or SEQ ID NO: 13. In some embodiments, the invention provides a nucleic acid sequence having at least 85% identity with the nucleic acid sequence of SEQ ID NO: 10 or SEQ ID NO: 13. In some embodiments, the invention provides a nucleic acid sequence having at least 90% identity with the nucleic acid sequence of SEQ ID NO: 10 or SEQ ID NO: 13. In some embodiments, the invention provides a nucleic acid sequence having at least 95% identity with the nucleic acid sequence of SEQ ID NO: 10 or SEQ ID NO: 13. In some embodiments, the nucleic acid sequence of the invention encodes an FGF polypeptide with increased bioactivity, solubility and/or stability (for example, increased thermostability) compared to the FGF2 polypeptide of SEQ ID NO: 1.

In some embodiments, the invention provides a nucleic acid sequence having at least 80% identity with the nucleic acid sequence of SEQ ID NO: 10. In some embodiments, the invention provides a nucleic acid sequence having at least 85% identity with the nucleic acid sequence of SEQ ID NO: 10. In some embodiments, the invention provides a nucleic acid sequence having at least 90% identity with the nucleic acid sequence of SEQ ID NO: 10. In some embodiments, the invention provides a nucleic acid sequence having at least 95% identity with the nucleic acid sequence of SEQ ID NO: 10. In some embodiments, the nucleic acid sequence of the invention encodes an FGF polypeptide with increased bioactivity, solubility and/or stability (for example, increased thermostability) compared to the FGF2 polypeptide of SEQ ID NO: 1.

In some embodiments, the invention provides a nucleic acid sequence having at least 80% identity with the nucleic acid sequence of SEQ ID NO: 13. In some embodiments, the invention provides a nucleic acid sequence having at least 85% identity with the nucleic acid sequence of SEQ ID NO: 13. In some embodiments, the invention provides a nucleic acid sequence having at least 90% identity with the nucleic acid sequence of SEQ ID NO: 13. In some embodiments, the invention provides a nucleic acid sequence having at least 95% identity with the nucleic acid sequence of SEQ ID NO: 13. In some embodiments, the nucleic acid sequence of the invention encodes an FGF polypeptide with increased bioactivity, solubility and/or stability (for example, increased thermostability)compared to the FGF2 polypeptide of SEQ ID NO: 1.

In a preferred embodiment, the invention provides a nucleic acid sequence having at least 95% identity with the nucleic acid sequence of any one of SEQ ID NOs: 8-13, which encodes an FGF polypeptide with increased stability (such as thermostability) compared to the FGF2 polypeptide of SEQ ID NO: 1.

In a preferred embodiment, the invention provides a nucleic acid sequence having at least 95% identity with the nucleic acid sequence of SEQ ID NO: 10 or SEQ ID NO: 13, which encodes an FGF polypeptide with increased stability (such as thermostability) compared to the FGF2 polypeptide of SEQ ID NO: 1.

In a preferred embodiment, the invention provides a nucleic acid sequence having at least 95% identity with the nucleic acid sequence of SEQ ID NO: 10, which encodes an FGF polypeptide with increased bioactivity compared to the FGF2 polypeptide of SEQ ID NO: 1.

In a preferred embodiment, the invention provides a nucleic acid sequence having at least 95% identity with the nucleic acid sequence of SEQ ID NO: 10, which encodes an FGF polypeptide with increased stability (such as thermostability) compared to the FGF2 polypeptide of SEQ ID NO: 1.

In a preferred embodiment, the invention provides a nucleic acid sequence having at least 95% identity with the nucleic acid sequence of SEQ ID NO: 13, which encodes an FGF polypeptide with increased bioactivity compared to the FGF2 polypeptide of SEQ ID NO: 1.

In a preferred embodiment, the invention provides a nucleic acid sequence having at least 95% identity with the nucleic acid sequence of SEQ ID NO: 13, which encodes an FGF polypeptide with increased stability (such as thermostability) compared to the FGF2 polypeptide of SEQ ID NO: 1.

The nucleic acid molecule of the invention can be mRNA, DNA, cDNA or genomic DNA.

In some embodiments, the invention provides a nucleic acid sequence that hybridises with the complement of the nucleic acid sequence of any one of SEQ ID NOs: 8-13.

In some embodiments, the invention provides a nucleic acid sequence that hybridises with the complement of the nucleic acid sequence of SEQ ID NO: 10 or SEQ ID NO: 13.

In some embodiments, the invention provides a nucleic acid sequence that hybridises with the complement of the nucleic acid sequence of SEQ ID NO: 10.

In some embodiments, the invention provides a nucleic acid sequence that hybridises with the complement of the nucleic acid sequence of SEQ ID NO: 13.

In some embodiments, the invention provides a nucleic acid sequence which is the reverse complement of a nucleic acid sequence of the invention.

In some embodiments, the invention provides a vector comprising a polynucleotide of the present invention.

In some embodiments, the invention provides a cell comprising the polypeptide or nucleic acid sequence described herein. The cell may be genetically engineered to express the vector of the invention.

In some embodiments, the cell is a bacteria cell, a yeast cell, a plant cell, an insect cell or a mammalian cell. In some embodiments, the bacteria cell is an Escherichia coli (E. coli) cell or a Corynebacterium glutamicum cell. In some embodiments, the cell is an E. coli cell. In some embodiments, the cell is a yeast cell.

In some embodiments, the invention provides use of the polypeptide described herein in a growth media. In some embodiments, the growth media is an animal cell growth media. The growth media may be serum-free.

A growth media is a media used for the viability, growth and/or storage of cells. In some embodiments, the growth media of the invention is used for culture of fibroblasts, myoblasts, adipocytes, mesenchymal stem cells or iPSCs.

A growth media of the invention can additionally comprise one or more additional growth factors, serum or serum replacement, one or more hormones, one or more antibiotics, one or more trace elements and/or one or more antioxidants.

In some embodiments, the invention provides a method of growing a cell, wherein the method comprises cultivating the cell in a culture medium containing the polypeptide described herein. In some embodiments, the cell is an animal cell. In some embodiments the growth media is an animal growth media.

In some embodiments, the invention provides a method of preparing a polypeptide described herein. In some embodiments, the method comprises recombinant production and synthesis of a polypeptide described herein. In some embodiments, the method is a cell-free method or a method which utilises cells.

In some embodiments, the method comprises cultivating a cell described herein under conditions which allow for expression of a polypeptide described herein and, optionally, recovering the expressed polypeptide.

In some embodiments, the method comprises cell-free protein synthesis.

In some embodiments, the method comprises i) culturing a nucleic acid sequence of the invention in a cell lysate with ribosomal activity and ii) synthesising a protein encoded by the nucleic acid with a cell-free protein synthesis reaction.

Cell-free protein synthesis is described, for example, in Gregorio et al, “A User's Guide to Cell-Free Protein Synthesis”, Methods Protoc., 2019, the contents of which are incorporated by reference.

It will be understood that particular embodiments described herein are shown by way of illustration and not as limitations of the invention. The principal features of this invention can be employed in various embodiments without departing from the scope of the invention. Those skilled in the art will recognise, or be able to ascertain using no more than routine study, numerous equivalents to the specific procedures described herein. Such equivalents are considered to be within the scope of this invention and are covered by the claims. All publications mentioned in the specification are indicative of the level of skill of those skilled in the art to which this invention pertains.

All publications are herein incorporated by reference to the same extent as if each individual publication was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the inherent variation of error for the feature in the below.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the polypeptides, nucleic acids and media disclosed and claimed herein can be made and executed without undue experimentation in light of the present disclosure. While the invention have been described in terms of preferred embodiments, it will be apparent to those of skill in the art that variations may be applied to the polypeptides, nucleic acids, media and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The invention can be further described in the following numbered clauses:

1. A fibroblast growth factor (FGF) polypeptide comprising at least one modification selected from the group consisting of an amino acid substitution, an amino acid deletion, an amino acid insertion, and combinations thereof relative to the sequence set forth in SEQ ID NO: 1, wherein the modified FGF polypeptide exhibits increased thermostability compared to the FGF2 polypeptide of SEQ ID NO: 1.

2. The FGF polypeptide of clause 1, wherein the at least one modification comprises a substitution of histidine at position 34 of SEQ ID NO: 1 and/or a substitution of aspartic acid at position 89 of SEQ ID NO: 1.

3. The FGF polypeptide of clause 2, wherein the substitution at position 34 of SEQ ID NO: 1 is an H34E substitution and/or the substitution at position 89 of SEQ ID NO: 1 is an D89E substitution.

4. The FGF polypeptide of any one of clauses 1 to 3, comprising the amino acid sequence:

GFFLRIEXaa₃₈Xaa₃₉Xaa₄₀Xaa₄₁Xaa₄₂Xaa₄₃Xaa₄₄Xaa₄₅Xaa₄₆
Xaa₄₇Xaa₄₈Xaa₄₉Xaa₅₀Xaa₅₁Xaa₅₂Xaa₅₃Xaa₅₄Xaa₅₅Xaa₅₆KLQLQ
AEERXaa₆₆GVVXaa₇₀IKGVXaa₇₅Xaa₇₆Xaa₇₇Xaa₇₈YLAMKEDGRL
Xaa₈₉Xaa₉₀Xaa₉₁Xaa₉₂Xaa₉₃Xaa₉₄Xaa₉₅Xaa₉₆EEC

wherein:
Xaa₃₈ is P, D or a deletion, Xaa₃₉ is P or a deletion, Xaa₄₀ is D or G, Xaa₄₁ is G or a deletion, Xaa₄₂ is R or a deletion, Xaa₄₃ is V or a deletion, Xaa₄₄ is D, E or K, Xaa₄₅ is G, D or V, Xaa₄₆ is V, A or D, Xaa₄₇ is G or a deletion, Xaa₄₈ is R or V, Xaa₄₉ is E or R, Xaa₅₀ is K or E, Xaa₅₁ is K, S or a deletion, Xaa₅₂ is Q, S, D or a deletion, Xaa₅₃ is D or P, Xaa₅₄ is P, D or a deletion, Xaa₅₅ is H or a deletion, Xaa₅₆ is I or a deletion, Xaa₆₆ is G or a deletion, Xaa₇₀ is L, S or T, Xaa₇₅ is L, P or a deletion, Xaa₇₆ is A, D or a deletion, Xaa₇₇ is N, L or I, Xaa₇₆ is R, K or a deletion, Xaa₈₉ is L or F, Xaa₉₀ is Y, A or a deletion, Xaa₉₁ is A, L or E, Xaa₉₂ is L, S or a deletion, Xaa₉₃ is E, K, P, A or a deletion, Xaa₉₄ is C, L, E or a deletion, Xaa₉₅ is P or A, Xaa₉₆ is E, T or a deletion.

5. The FGF polypeptide of any one of the preceding clauses, wherein the polypeptide comprises a sequence of any one of SEQ ID NOs: 2-7, or a sequence with at least 80%, 85%, 90% or 95% sequence similarity to any one of SEQ ID NOs: 2-7, wherein the polypeptide has a melting temperature of at least 55° C.

6. The FGF polypeptide of any one of clauses 1 to 5, wherein the polypeptide comprises a sequence of SEQ ID NO: 2 or SEQ ID NO: 3 or a sequence with at least 80%, 85%, 90% or 95% sequence similarity to SEQ ID NO: 2 or SEQ ID NO: 3 wherein the polypeptide has a melting temperature of at least 55° C.

7. The FGF polypeptide of any one of clauses 1 to 5, wherein the polypeptide comprises a sequence of SEQ ID NO: 4 or SEQ ID NO: 5 or a sequence with at least 80%, 85%, 90% or 95% sequence similarity to SEQ ID NO: 4 or SEQ ID NO: 5, wherein the polypeptide has a melting temperature of at least 55° C.

8. The FGF polypeptide of any one of clauses 1 to 5, wherein the polypeptide comprises a sequence of SEQ ID NO: 6 or SEQ ID NO: 7 or a sequence with at least 80%, 85%, 90% or 95% sequence similarity to SEQ ID NO: 6 or SEQ ID NO: 7, wherein the polypeptide has a melting temperature of at least 55° C.

9. The FGF polypeptide of any one of the preceding clauses, wherein the polypeptide is soluble and/or capable of binding a fibroblast growth factor receptor (FGFR), optionally FGFR2.

10. A nucleic acid comprising a sequence selected from the group consisting of:
(a) a nucleic acid sequence encoding an FGF polypeptide of any preceding clause;
(b) a nucleic acid sequence having at least 80% similarity with the nucleic acid sequence of any one of SEQ ID NOs: 8-13 and encoding an FGF polypeptide of any preceding clause;
(c) a nucleic acid sequence hybridizing with the complement of the nucleic acid sequence of any one of SEQ ID NOs: 8-13 and encoding an FGF polypeptide of any preceding clause; or
(d) a nucleic acid sequence which is the reverse complement of a nucleic acid sequence as defined in (a), (b) or (c).

11. A cell comprising the FGF polypeptide or nucleic acid sequence of any preceding clause.

12. The cell of clause 11, wherein the cell is a bacteria cell, a yeast cell, a plant cell, an insect cell or a mammalian cell.

13. A method of preparing an FGF polypeptide according to any one of clauses 1-9, wherein the method comprises cultivating the cell of clause 11 or clause 12 under conditions which allow for expression of said polypeptide and, optionally, recovering the expressed polypeptide.

14. A method of preparing an FGF polypeptide according to any one of clauses 1-9, wherein the method comprises i) culturing a nucleic acid sequence of clause 10 in a cell lysate with ribosomal activity and ii) synthesising a protein encoded by the nucleic acid with a cell-free protein synthesis reaction.

15. A method of growing an animal cell, wherein the method comprises cultivating the animal cell in a growth media containing the FGF polypeptide of any one of clauses 1-9.

16. Use of the FGF polypeptide of any one of clauses 1-9 in an animal growth media.

The present invention is described in more detail in the following non limiting exemplification.

### EXAMPLES

The following examples will be useful in demonstrating the present invention.

### Example 1: Polypeptide Variants and Alignments

The following 6 variants are disclosed herein:

Variant 1 (SEQ ID NO: 2):
AEIPEDGGSGAFPPGHFKDPKRLYGKNGGFFLRIEPDGRVEDAGVRESDPHIKLQLQAEERGV
VLIKGVLANRYLAMKEDGRLLALKLPEEECLFLEREEENHYNTYRSDKYPDWYVALKRTGQYKL
GPKTGPGQKAILFLPMSAKS
Variant 2 (SEQ ID NO: 3):
ALPEDGGSGAFPPGHFKDPKLLVGKNGGFFLRIEDGKVDGVRESDPHIKLQLQAEERGVVLIK
GVLANRYLAMKEDGRLLALKLPEEECFFERLEEENHYNTYRSDKKYPDWYVALKRTGQYKLGP
KTGPGQKAILFLPMSAKS
Variant 3 (SEQ ID NO: 4):
ALPEDGGSGAFPPGHFKDPKLLVDDGGFFLRIEPDGRVDGVREKSDPHIKLQLQAEERGVVLIK
GVLANRYLAMKEDGRLLYASKLPEEECFFFERLEEENHYNTYSDKYPDWYVALKRTGQYKLGP
KTGPGQKAILFLPMSAKS
Variant 4 (SEQ ID NO: 5):
ALPEDGGSGAFPPGHFKDPKLLVDDGGFFLRIEPDGRVDGVREKSDPHIKLQLQAEERGVVLIK
GVLANRYLAMKEDGRLFALPEATEECFFFERLEEENHYNTYSDKYPDWYVALKRTGQYKLGPK
TGPGQKAILFLPMSAKS
Variant 7 (SEQ ID NO: 6):
AEPEADGGAGAQPPGPDDGAEGGFFLRIEPPDGRVEVDGVRESDPHIKLQLQAEERGVVSIK
GVLKYLAMKEDGRLLYELALPEEECLERLEENHYNTYISDKKYPDPDWYVALKIKGQYELTPKT
GPGQKAILFLPMSAKS
Variant 9 (SEQ ID NO: 7):
AEPEADGGAGAQPPGPDDGAEGFFLRIEPPDGRVEVDGVRESDPDKLQLQAEERGGVVTIKG
VPDIYLAMKEDGRLLYELALPEEECLERLEENNYNTYSDKKYPDPDWYVALKIKTGQYELTGPK
TGPPDGQKAILFLPMSAKS

The nucleic acid sequences equivalent to SEQ ID NOs: 2-7 (variants 1-4, 7 and 9) are provided as SEQ ID NOs: 8-13 and shown below:

Variant 1 (SEQ ID NO: 8):
GCGGAAATTCCGGAAGATGGCGGCAGCGGCGCGTTTCCGCCGGGCCATTTTAAAGATCC
GAAACGCCTGTATGGCAAAAACGGCGGCTTTTTTCTGCGCATTGAACCGGATGGCCGCGT
GGAAGATGCGGGCGTGCGCGAAAGCGATCCGCATATTAAACTGCAGCTGCAGGCGGAAG
AACGCGGCGTGGTGCTGATTAAAGGCGTGCTGGCGAACCGCTATCTGGCGATGAAAGAAG
ATGGCCGCCTGCTGGCGCTGAAACTGCCGGAAGAAGAATGCCTGTTTCTGGAACGCGAAG
AAGAAAACCATTATAACACCTATCGCAGCGATAAATATCCGGATTGGTATGTGGCGCTGAA
ACGCACCGGCCAGTATAAACTGGGCCCGAAAACCGGCCCGGGCCAGAAAGCGATTCTGTT
TCTGCCGATGAGCGCGAAAAGC
Variant 2 (SEQ ID NO: 9):
GCGCTGCCGGAAGATGGCGGCAGCGGCGCGTTTCCGCCGGGCCATTTTAAAGATCCGAA
ACTGCTGGTGGGCAAAAACGGCGGCTTTTTTCTGCGCATTGAAGATGGCAAAGTGGATGG
CGTGCGCGAAAGCGATCCGCATATTAAACTGCAGCTGCAGGCGGAAGAACGCGGCGTGG
TGCTGATTAAAGGCGTGCTGGCGAACCGCTATCTGGCGATGAAAGAAGATGGCCGCCTGC
TGGCGCTGAAACTGCCGGAAGAAGAATGCTTTTTTGAACGCCTGGAAGAAGAAAACCATTA
TAACACCTATCGCAGCGATAAAAAATATCCGGATTGGTATGTGGCGCTGAAACGCACCGGC
CAGTATAAACTGGGCCCGAAAACCGGCCCGGGCCAGAAAGCGATTCTGTTTCTGCCGATG
AGCGCGAAAAGC
Variant 3 (SEQ ID NO: 10):
GCGCTGCCGGAAGATGGCGGCAGCGGCGCGTTTCCGCCGGGCCATTTTAAAGATCCGAA
ACTGCTGGTGGATGATGGCGGCTTTTTTCTGCGCATTGAACCGGATGGCCGCGTGGATGG
CGTGCGCGAAAAAAGCGATCCGCATATTAAACTGCAGCTGCAGGCGGAAGAACGCGGCGT
GGTGCTGATTAAAGGCGTGCTGGCGAACCGCTATCTGGCGATGAAAGAAGATGGCCGCCT
GCTGTATGCGAGCAAACTGCCGGAAGAAGAATGCTTTTTTTTTGAACGCCTGGAAGAAGAA
AACCATTATAACACCTATAGCGATAAATATCCGGATTGGTATGTGGCGCTGAAACGCACCG
GCCAGTATAAACTGGGCCCGAAAACCGGCCCGGGCCAGAAAGCGATTCTGTTTCTGCCGA
TGAGCGCGAAAAGC
Variant 4 (SEQ ID NO: 11):
GCGCTGCCGGAAGATGGCGGCAGCGGCGCGTTTCCGCCGGGCCATTTTAAAGATCCGAA
ACTGCTGGTGGATGATGGCGGCTTTTTTCTGCGCATTGAACCGGATGGCCGCGTGGATGG
CGTGCGCGAAAAAAGCGATCCGCATATTAAACTGCAGCTGCAGGCGGAAGAACGCGGCGT
GGTGCTGATTAAAGGCGTGCTGGCGAACCGCTATCTGGCGATGAAAGAAGATGGCCGCCT
GTTTGCGCTGCCGGAAGCGACCGAAGAATGCTTTTTTTTTGAACGCCTGGAAGAAGAAAAC
CATTATAACACCTATAGCGATAAATATCCGGATTGGTATGTGGCGCTGAAACGCACCGGCC
AGTATAAACTGGGCCCGAAAACCGGCCCGGGCCAGAAAGCGATTCTGTTTCTGCCGATGA
GCGCGAAAAGC
Variant 7 (SEQ ID NO: 12):
GCGGAACCGGAAGCGGATGGGGGCGCGGGAGCCCAACCACCTGGTCCTGACGATGGTG
CTGAGGGCGGTTTTTTTCTGCGCATTGAACCGCCGGATGGCCGCGTGGAAGTGGATGGC
GTGCGCGAAAGCGATCCGCATATTAAACTGCAGCTGCAGGCGGAAGAACGCGGCGTGGT
GAGCATTAAAGGCGTGCTGAAATATCTGGCGATGAAAGAAGATGGCCGCCTGCTGTATGA
ACTGGCGCTGCCGGAAGAAGAATGCCTGGAACGCCTGGAAGAAAACCATTATAACACCTA
TATTAGCGATAAAAAATATCCGGATCCGGATTGGTATGTGGCGCTGAAAATTAAAGGCCAG
TATGAACTGACCCCGAAAACCGGCCCGGGCCAGAAAGCGATTCTGTTTCTGCCGATGAGC
GCGAAAAGC
Variant 9 (SEQ ID NO: 13):
GCGGAACCGGAAGCGGATGGCGGCGCGGGCGCGCAGCCGCCGGGCCCGGATGATGGC
GCGGAAGGCTTTTTTCTGCGCATTGAACCGCCGGATGGCCGCGTGGAAGTGGATGGCGT
GCGCGAAAGCGATCCGGATAAACTGCAGCTGCAGGCGGAAGAACGCGGCGGCGTGGTGA
CCATTAAAGGCGTGCCGGATATTTATCTGGCGATGAAAGAAGATGGCCGCCTGCTGTATGA
ACTGGCGCTGCCGGAAGAAGAATGCCTGGAACGCCTGGAAGAAAACAACTATAACACCTA
TAGCGATAAAAAATATCCGGATCCGGATTGGTATGTGGCGCTGAAAATTAAAACCGGCCAG
TATGAACTGACCGGCCCGAAAACCGGCCCGCCGGATGGCCAGAAAGCGATTCTGTTTCTG
CCGATGAGCGCGAAAAGC

Alignments were generated using the Clustal Omega Multiple Sequence Alignment tool from EMBL-EBI. Version 0(1.2.4) was used, retrieved from “https://www.ebi.ac.uk/Tools/msa/clustalo/”.

Alignment of the amino acid sequence of variants 1-4, 7 and 9 (SEQ ID NOs: 2-7) is shown in FIG. 1.

Alignment of the amino acid sequence of variants 1-4, 7 and 9 (SEQ ID NOs: 2-7) and the wild-type FGF2 sequence (SEQ ID NO: 1) is shown in FIG. 2.

A number of differences can be seen that all 6 variants have in common, but that vary from the wild-type FGF2, such as the H34E substitution and the D89E substitution.

### Example 2: Polypeptide Expression

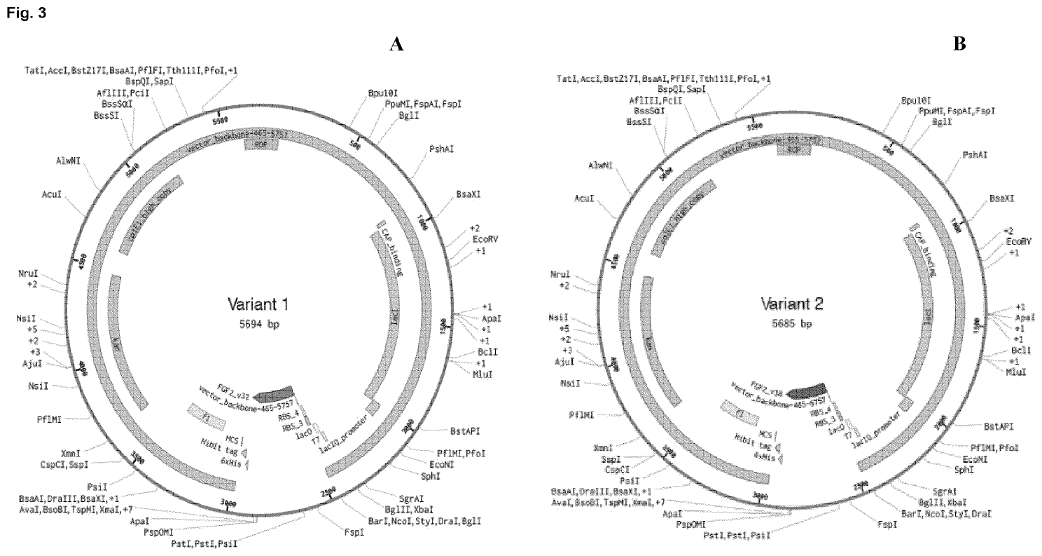

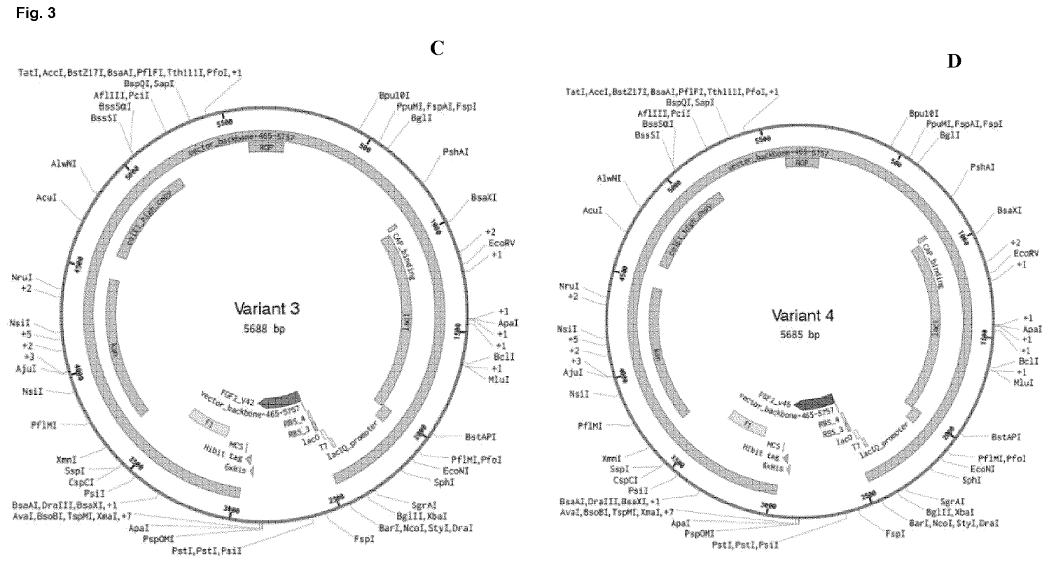

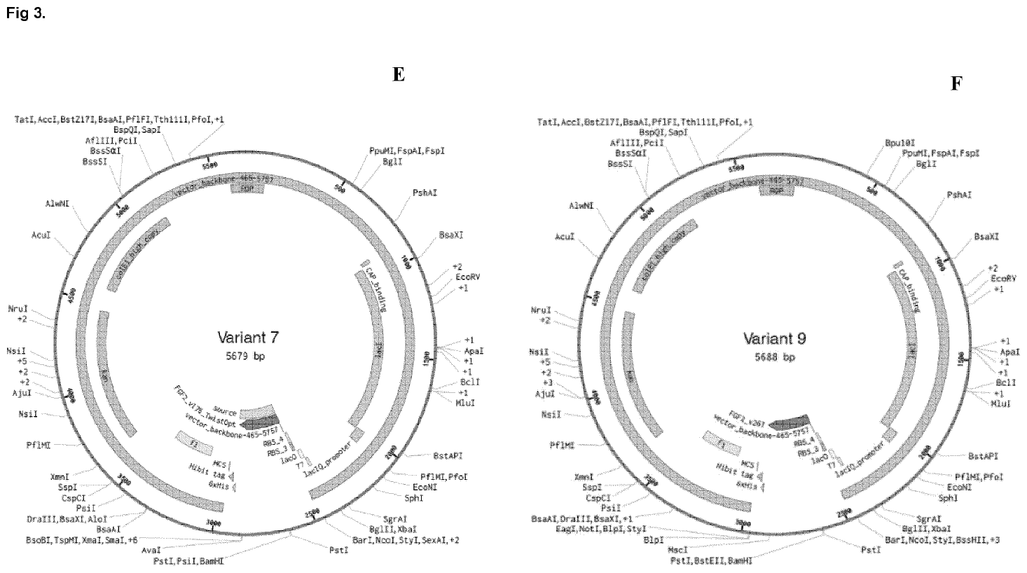

Plasmid inserts for each of the 6 variants of SEQ ID NOs: 2-7 were generated, by converting primary amino acid sequences to DNA sequences via codon optimisation. These plasmids are shown in FIGS. 3A-3F.

The insert containing the engineered variants were digested with Ncol and Xhol and ligated into a pET-28a (+) backbone. E. Coli strain BL21(DE3) was used to transform 5 μl of the reaction mix. Electroporation was carried out using the protocols provided for the MicroPulser Electroporator (Bio-Rad). Single colonies were picked, and the sequence was verified via Sanger sequencing (Azenta).

Overnight culture was prepared using 10 ml of LB supplemented with 50 μg/ml of Kanamycin. The starter culture was diluted 1:100 into 50 ml of autoinducible expression media (Magic Media, Invitrogen) for 24 hours, 250 rpm at 30° C. The culture was pelleted at 10,000×g for 10 min at 4° C. and stored at −20° C.

Lysis was carried out on in buffer A (50 mM Tris, 300 mM NaCl, pH 8) supplemented with 5% Glycerol and 1 mM DTT, 10 mg/ml lysozyme and 1 U/ml DNasel (NEB) and protease inhibitor cocktail (Roche). Sonication (Branson Ultrasonics) was performed at 45%, at 10 seconds on/off. The lysate was pelleted at 10,000×g for 30 minutes at 4° C.

Ni-charged magnetic beads (GenScript) were pre-equilibrated with BufferA supplemented with 10 mM Imidazole, and were incubated with the lysate at 4° C. prior to affinity chromatography. Buffer A supplemented with 50 mM Imidazole was used to wash the beads for 10 column volumes, three times. Buffer A supplemented with 300 mM Imidazole was used to elute the proteins in 5 column volumes of magnetic resin. 10 μl of eluant was analysed with SDS-PAGE (40 mA, 60 min, Bis-Tris/MES). The process was carried out on ice.

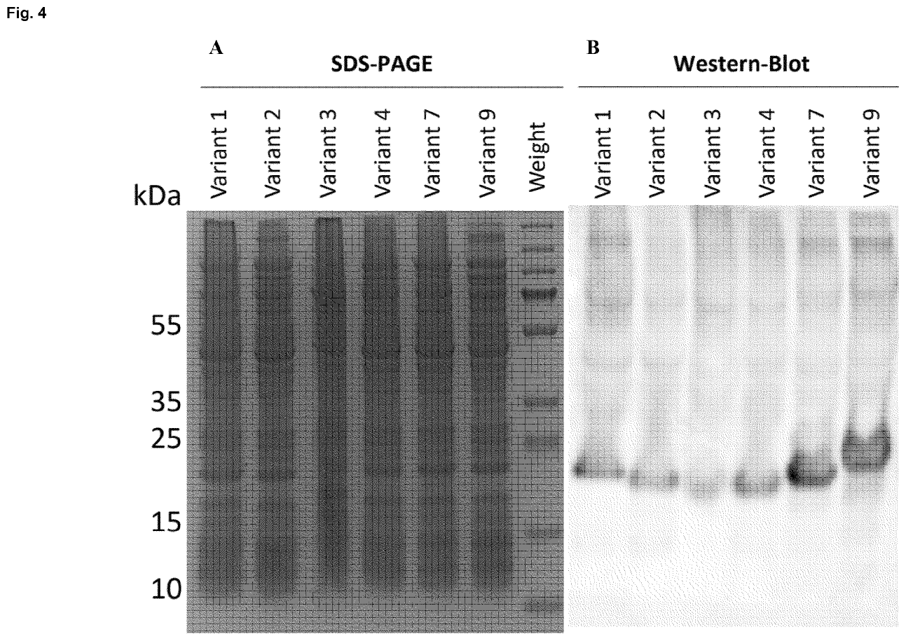

The results of the SDS-PAGE are shown in FIG. 4A.

Approximately 20 μg of protein was loaded on Bis-Tris gel (GenScript), then blotted (100V, 1 hour, 4° C.) on to a nitrocellulose membrane using the Trans-Blot module (Bio-rad). The membrane was blocked with TBS supplemented with 5% skimmed milk powder. Variants were detected using a 1:1000 dilution of the Mouse anti-HiBiT primary antibody (Promega) followed by 1:2500 dilution of the horseradish peroxidase (HRP) conjugated anti-mouse secondary antibody (Promega). Bands were identified using the ECL Western Blotting Substrate (Promega) as per manufacturer instructions.

The results of the western blot are shown in FIG. 4B.

As can be seen from FIGS. 4A and 4B, all variants are successfully expressed.

In order to quantify protein expression, the eluant was diluted 1:1000 for quantification using Nano-Glo kit (Promega). 5 μl of diluted protein was treated with 5 μl of reaction mix and relative luminescence was plotted against a standard curve using a HiBiT control protein (Promega).

The results of the HiBiT analysis are shown in Table 1.

TABLE 1
Results of nano-glo HiBiT protein quantification.

| Variant | ng/μl |
|---|---|
| Variant 1 | 12.600 |
| SEQ ID NO: 2 | |
| Variant 2 | 9.135 |
| SEQ ID NO: 3 | |
| Variant 3 | 83.356 |
| SEQ ID NO: 4 | |
| Variant 4 | 6.081 |
| SEQ ID NO: 5 | |
| Variant 7 | 46.473 |
| SEQ ID NO: 6 | |
| Variant 9 | 198.437 |
| SEQ ID NO: 7 | |

As can be seen from Table 1, all variants show protein expression, with variant 9 showing the greatest expression, followed by variant 3.

### Example 3: Polypeptide Bioactivity

Receptor binding was assessed using ADP-Glo kinase assay (Promega), with a commercial FGF2 (Qkine) as a positive control. A serial dilution was performed with the eluant in Reaction Buffer A (40 mM Tris, pH 7.5, 20 mM MgCl2, 0.1 mg/ml BSA). 1 μl of dilution was used in a 5 μl reaction with 0.2 μg/μl of E4Y1 substrate, 3ng FGFR2 receptor and 50 μM ATP. Relative luminescence was measured to be plotted against a standard to calculate the degree of ATP to ADP conversion and plotted against log concentration for a dose response curve in software Graphpad Prism.

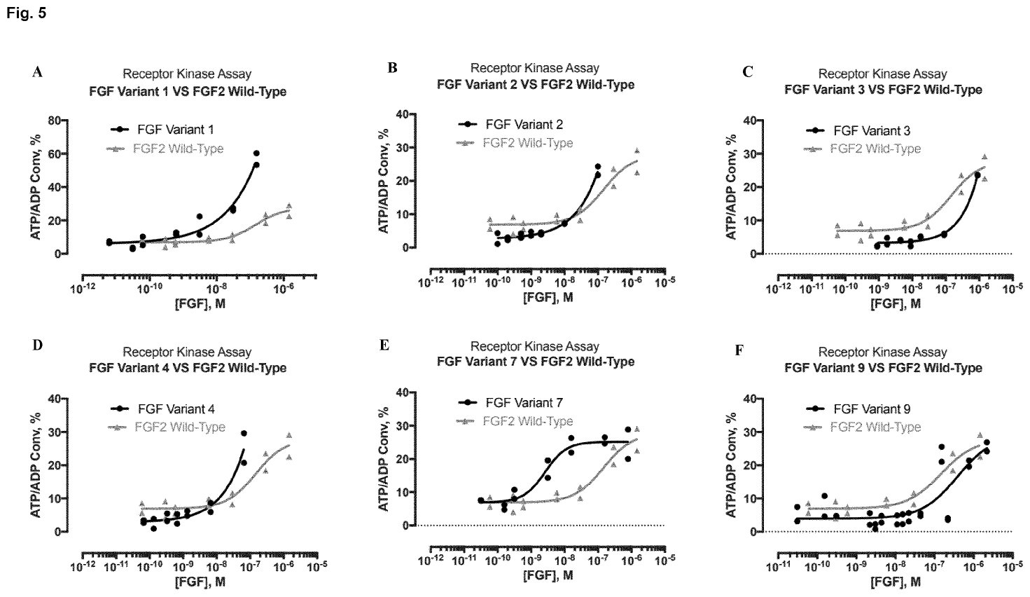

The results are shown in FIGS. 5A-5F.

As can be seen from the graphs, all the variants presented show bioactivity. The bioactivity is comparable to that of wild-type FGF2. Some variants, such as variant 1 and variant 7, show higher affinity for the receptor (i.e., enhanced bioactivity) compared to wild-type FGF2.

### Example 4: Polypeptide Bioactivity—ONE-Glo

Subsequently, bioactivity was further confirmed using a more sensitive assay, the ONE-Glo Luciferase assay (Promega). This assay also has the benefit of being a direct measure of protein functionality.

HEK293T cells were seeded at 10,000 cells/well in a clear, flat bottom 96-well plate and transfected with pGL4.33 (Promega) containing a Serum Response element (SRE) at 2:1 ratio with Viafect transfection reagent (Promega) as per manufacturer's instructions. Cells were starved overnight in DMEM-F12 supplemented with 0.5% FBS and 1% Penicillin Streptomycin. Elution's of purified protein variants were serially diluted in DMEM-F12 supplemented with 1% Penicillin streptomycin and treated for 6 hours with DMEM-F12 supplemented with 20% FBS+20 ng/ml PMA as positive control and DMEM-F12 as negative control. One-Glo buffer and substrate was reconstituted and added to the samples as per manufacturer's instructions. All experiments were performed in triplicate.

For accurate quantitation, 10 μl of the diluted samples were diluted in 90 μl DMEM F12 and analysed using the His-Tag ELISA Detection kit (GenScript) as per manufacturer's instructions. Luminescence of the samples were measured. Relative luminescence intensities (RLU) were normalized using the negative control. Dose response curves were plotted and EC50 values were calculated using the Graphpad—Prism software.

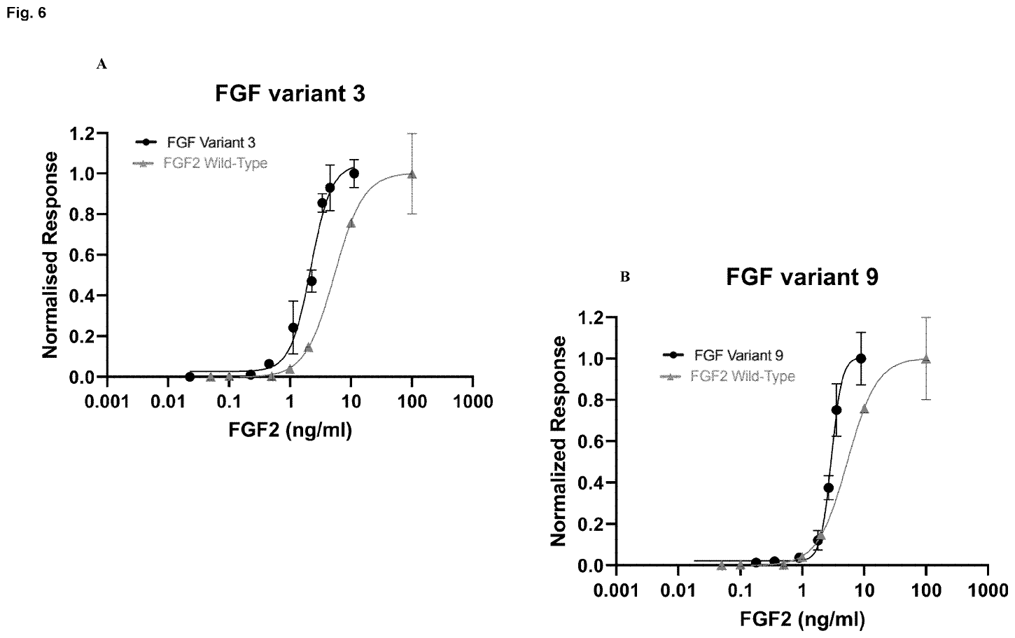

The results are shown in FIGS. 6A and 6B.

As can be seen from the graphs, both variant 3 and variant 9 show improved bioactivity compared to wild-type FGF2 using the more sensitive One-Glo Luciferase assay.

### Example 5: Polypeptide Solubility—SDS-PAGE and Western Blot

To assess solubility, the insoluble pellet at the end of the lysis step above was collected. The pellet was resuspended in IB Wash buffer (20 mM Tris-HCl, 50 mM NaCl, 10% Glycerol, 2M Urea, 1% Triton X-100, 50 mM beta-mercaptaethanol, pH 8.5) and pelleted (10,000 g, 5 min, 4° C.). The wash step was repeated twice. The resultant pellet was dissolved in equal volume IB Buffer (20 mM Tris-HCl, 50 mM NaCl, 10% Glycerol, 6M Urea, 1% Triton X-100, pH 10.5, supplemented with Complete mini tablet (Roche) as per manufacturer's instructions) as the lysis buffer. Both lysate and dissolved pellet was analysed using SDS-PAGE and western blot. The ratio of soluble to insoluble protein was calculated by quantifying the intensity of the 18 kDa band in both fractions using ImageJ.

The ratio was calculated using the formula:

$$Soluble fraction ( \% ) = \frac{{Intensity}_{Soluble}}{{Intensity}_{Soluble} + {Intensity}_{Insoluble}} \times 100$$

The results are shown in Table 2.

TABLE 2
Results of solubility analysis for the FGF polypeptides of SEQ
ID NOs: 2-7 and the wild-type FGF2 sequence (SEQ ID NO: 1).

| | Intensity (RLU) | | Fraction (%) | |
|---|---|---|---|---|
| Variant | Lysate | Pellet | Soluble | Insoluble |
| Variant 1 | 24173.83 | 51387.53 | 31.99232 | 68.00768 |
| SEQ ID NO: 2 | | | | |
| Variant 2 | 5730.137 | 0 | 100 | 0 |
| SEQ ID NO: 3 | | | | |
| Variant 3 | 10313.81 | 349.799 | 96.71969 | 3.280306 |
| SEQ ID NO: 4 | | | | |
| Variant 4 | 20629.32 | 4027.761 | 83.66489 | 16.33511 |
| SEQ ID NO: 5 | | | | |
| Variant 7 | 68059.02 | 69981.63 | 49.30361 | 50.69639 |
| SEQ ID NO: 6 | | | | |
| Variant 9 | 31508.37 | 0 | 100 | 0 |
| SEQ ID NO: 7 | | | | |
| WT | 75619.27 | 71767.77 | 51.30659 | 48.69341 |
| SEQ ID NO: 1 | | | | |

Measurements of zero in Table 2 indicate levels beyond the detection limit.

Table 2 shows all variants to be soluble. All variants show solubility at least comparable to wild-type FGF2. Some variants, variants 2, 3, 4 and 9, show greatly improved solubility compared to wild-type FGF2. Variants 3 and 9 show the particularly improved solubility compared to wild-type FGF2.

### Example 6: Polypeptide Thermostability

Thermostability of the polypeptides was predicted using property predictors trained on public datasets of FGF2 variants.

The results are shown in Table 3.

TABLE 3
Results of in silico thermostability predictions
for the FGF polypeptides of SEQ ID NOs: 2-7 and
the wild-type FGF2 sequence (SEQ ID NO: 1).

| | Thermostability |
|---|---|
| Variant | (melting temperature) (° C.) |
| Variant 1 | 65.015 |
| SEQ ID NO: 2 | |
| Variant 2 | 66.379 |
| SEQ ID NO: 3 | |
| Variant 3 | 66.469 |
| SEQ ID NO: 4 | |
| Variant 4 | 65.155 |
| SEQ ID NO: 5 | |
| Variant 7 | 78.696 |
| SEQ ID NO: 6 | |
| Variant 9 | 78.288 |
| SEQ ID NO: 7 | |
| WT | 55.016 |
| SEQ ID NO: 1 | |

All 6 variants also show increased thermostability compared to wild-type FGF2. The minimum increase is 10° C., with some variants showing even higher thermostability. The highest increase in thermostability is seen in variant 7, followed by variant 9. Variant 3 also shows an increase in thermostability compared to wild-type FGF2.

These data show that the polypeptides with the sequences of SEQ ID NOs: 2-7 have improved properties.

### Example 7: Thermostability—TSA Assay

Thermoshift Assay (TSA), or Differential Scanning Fluorometry (DSF) can be used to assess thermal stability. DSF was performed using the Protein Thermal Shift Dye kit (ThermoFisher). 12.5 μl of eluant was mixed with 2.5 μl Sypro-orange (8×) Dye and 5 μl Reaction buffer and the melting curve was plotted with Quantstudio 5 (ThermoFisher) from 25° C. to 99° C. with ramp rate 0.05/° C. The curve was fitted to a sigmoidal curve to extract melting temperature (Tm).

This assay can be used to further confirm the thermostability of the polypeptides of the invention.

SEQUENCES

| SEQ | | | |
|---|---|---|---|
| ID | | | |
| NO: | Description | Sequence | Length |
| 1 | Bovine wild- | ALPEDGGSGAFPPGHFKDPKRLYCKNGGFFLRIHPDGRV | 145 |
| | type FGF2 | DGVREKSDPHIKLQLQAEERGVVSIKGVCANRYLAMKED | |
| | (aa) | GRLLASKCVTDECFFFERLESNNYNTYRSRKYSSWYVAL | |
| | | KRTGQYKLGPKTGPGQKAILFLPMSAKS | |
| 2 | Variant 1 (aa) | AEIPEDGGSGAFPPGHFKDPKRLYGKNGGFFLRIEPDGR | 147 |
| | | VEDAGVRESDPHIKLQLQAEERGVVLIKGVLANRYLAMKE | |
| | | DGRLLALKLPEEECLFLEREEENHYNTYRSDKYPDWYVA | |
| | | LKRTGQYKLGPKTGPGQKAILFLPMSAKS | |
| 3 | Variant 2 (aa) | ALPEDGGSGAFPPGHFKDPKLLVGKNGGFFLRIEDGKVD | 144 |
| | | GVRESDPHIKLQLQAEERGVVLIKGVLANRYLAMKEDGRL | |
| | | LALKLPEEECFFERLEEENHYNTYRSDKKYPDWYVALKRT | |
| | | GQYKLGPKTGPGQKAILFLPMSAKS | |
| 4 | Variant 3 (aa) | ALPEDGGSGAFPPGHFKDPKLLVDDGGFFLRIEPDGRVD | 145 |
| | | GVREKSDPHIKLQLQAEERGVVLIKGVLANRYLAMKEDGR | |
| | | LLYASKLPEEECFFFERLEEENHYNTYSDKYPDWYVALKR | |
| | | TGQYKLGPKTGPGQKAILFLPMSAKS | |
| 5 | Variant 4 (aa) | ALPEDGGSGAFPPGHFKDPKLLVDDGGFFLRIEPDGRVD | 144 |
| | | GVREKSDPHIKLQLQAEERGVVLIKGVLANRYLAMKEDGR | |
| | | LFALPEATEECFFFERLEEENHYNTYSDKYPDWYVALKRT | |
| | | GQYKLGPKTGPGQKAILFLPMSAKS | |
| 6 | Variant 7 (aa) | AEPEADGGAGAQPPGPDDGAEGGFFLRIEPPDGRVEVD | 142 |
| | | GVRESDPHIKLQLQAEERGVVSIKGVLKYLAMKEDGRLLY | |
| | | ELALPEEECLERLEENHYNTYISDKKYPDPDWYVALKIKG | |
| | | QYELTPKTGPGQKAILFLPMSAKS | |
| 7 | Variant 9 (aa) | AEPEADGGAGAQPPGPDDGAEGFFLRIEPPDGRVEVDG | 145 |
| | | VRESDPDKLQLQAEERGGVVTIKGVPDIYLAMKEDGRLLY | |
| | | ELALPEEECLERLEENNYNTYSDKKYPDPDWYVALKIKTG | |
| | | QYELTGPKTGPPDGQKAILFLPMSAKS | |
| 8 | Variant 1 (nt) | GCGGAAATTCCGGAAGATGGCGGCAGCGGCGCGTTTC | 441 |
| | | CGCCGGGCCATTTTAAAGATCCGAAACGCCTGTATGGC | |
| | | AAAAACGGCGGCTTTTTTCTGCGCATTGAACCGGATGG | |
| | | CCGCGTGGAAGATGCGGGCGTGCGCGAAAGCGATCC | |
| | | GCATATTAAACTGCAGCTGCAGGCGGAAGAACGCGGC | |
| | | GTGGTGCTGATTAAAGGCGTGCTGGCGAACCGCTATCT | |
| | | GGCGATGAAAGAAGATGGCCGCCTGCTGGCGCTGAAA | |
| | | CTGCCGGAAGAAGAATGCCTGTTTCTGGAACGCGAAGA | |
| | | AGAAAACCATTATAACACCTATCGCAGCGATAAATATCC | |
| | | GGATTGGTATGTGGCGCTGAAACGCACCGGCCAGTAT | |
| | | AAACTGGGCCCGAAAACCGGCCCGGGCCAGAAAGCGA | |
| | | TTCTGTTTCTGCCGATGAGCGCGAAAAGC | |
| 9 | Variant 2 (nt) | GCGCTGCCGGAAGATGGCGGCAGCGGCGCGTTTCCG | 432 |
| | | CCGGGCCATTTTAAAGATCCGAAACTGCTGGTGGGCAA | |
| | | AAACGGCGGCTTTTTTCTGCGCATTGAAGATGGCAAAG | |
| | | TGGATGGCGTGCGCGAAAGCGATCCGCATATTAAACTG | |
| | | CAGCTGCAGGCGGAAGAACGCGGCGTGGTGCTGATTA | |
| | | AAGGCGTGCTGGCGAACCGCTATCTGGCGATGAAAGA | |
| | | AGATGGCCGCCTGCTGGCGCTGAAACTGCCGGAAGAA | |
| | | GAATGCTTTTTTGAACGCCTGGAAGAAGAAAACCATTAT | |
| | | AACACCTATCGCAGCGATAAAAAATATCCGGATTGGTAT | |
| | | GTGGCGCTGAAACGCACCGGCCAGTATAAACTGGGCC | |
| | | CGAAAACCGGCCCGGGCCAGAAAGCGATTCTGTTTCT | |
| | | GCCGATGAGCGCGAAAAGC | |
| 10 | Variant 3 (nt) | GCGCTGCCGGAAGATGGCGGCAGCGGCGCGTTTCCG | 435 |
| | | CCGGGCCATTTTAAAGATCCGAAACTGCTGGTGGATGA | |
| | | TGGCGGCTTTTTTCTGCGCATTGAACCGGATGGCCGCG | |
| | | TGGATGGCGTGCGCGAAAAAAGCGATCCGCATATTAAA | |
| | | CTGCAGCTGCAGGCGGAAGAACGCGGCGTGGTGCTGA | |
| | | TTAAAGGCGTGCTGGCGAACCGCTATCTGGCGATGAAA | |
| | | GAAGATGGCCGCCTGCTGTATGCGAGCAAACTGCCGG | |
| | | AAGAAGAATGCTTTTTTTTTGAACGCCTGGAAGAAGAAA | |
| | | ACCATTATAACACCTATAGCGATAAATATCCGGATTGGT | |
| | | ATGTGGCGCTGAAACGCACCGGCCAGTATAAACTGGG | |
| | | CCCGAAAACCGGCCCGGGCCAGAAAGCGATTCTGTTT | |
| | | CTGCCGATGAGCGCGAAAAGC | |
| 11 | Variant 4 (nt) | GCGCTGCCGGAAGATGGCGGCAGCGGCGCGTTTCCG | 432 |
| | | CCGGGCCATTTTAAAGATCCGAAACTGCTGGTGGATGA | |
| | | TGGCGGCTTTTTTCTGCGCATTGAACCGGATGGCCGCG | |
| | | TGGATGGCGTGCGCGAAAAAAGCGATCCGCATATTAAA | |
| | | CTGCAGCTGCAGGCGGAAGAACGCGGCGTGGTGCTGA | |
| | | TTAAAGGCGTGCTGGCGAACCGCTATCTGGCGATGAAA | |
| | | GAAGATGGCCGCCTGTTTGCGCTGCCGGAAGCGACCG | |
| | | AAGAATGCTTTTTTTTTGAACGCCTGGAAGAAGAAAACC | |
| | | ATTATAACACCTATAGCGATAAATATCCGGATTGGTATG | |
| | | TGGCGCTGAAACGCACCGGCCAGTATAAACTGGGCCC | |
| | | GAAAACCGGCCCGGGCCAGAAAGCGATTCTGTTTCTG | |
| | | CCGATGAGCGCGAAAAGC | |
| 12 | Variant 7 (nt) | GCGGAACCGGAAGCGGATGGGGGCGCGGGAGCCCAA | 426 |
| | | CCACCTGGTCCTGACGATGGTGCTGAGGGCGGTTTTTT | |
| | | TCTGCGCATTGAACCGCCGGATGGCCGCGTGGAAGTG | |
| | | GATGGCGTGCGCGAAAGCGATCCGCATATTAAACTGCA | |
| | | GCTGCAGGCGGAAGAACGCGGCGTGGTGAGCATTAAA | |
| | | GGCGTGCTGAAATATCTGGCGATGAAAGAAGATGGCC | |
| | | GCCTGCTGTATGAACTGGCGCTGCCGGAAGAAGAATG | |
| | | CCTGGAACGCCTGGAAGAAAACCATTATAACACCTATA | |
| | | TTAGCGATAAAAAATATCCGGATCCGGATTGGTATGTG | |
| | | GCGCTGAAAATTAAAGGCCAGTATGAACTGACCCCGAA | |
| | | AACCGGCCCGGGCCAGAAAGCGATTCTGTTTCTGCCG | |
| | | ATGAGCGCGAAAAGC | |
| 13 | Variant 9 (nt) | GCGGAACCGGAAGCGGATGGCGGCGCGGGCGCGCAG | 435 |
| | | CCGCCGGGCCCGGATGATGGCGCGGAAGGCTTTTTTC | |
| | | TGCGCATTGAACCGCCGGATGGCCGCGTGGAAGTGGA | |
| | | TGGCGTGCGCGAAAGCGATCCGGATAAACTGCAGCTG | |
| | | CAGGCGGAAGAACGCGGCGGCGTGGTGACCATTAAAG | |
| | | GCGTGCCGGATATTTATCTGGCGATGAAAGAAGATGGC | |
| | | CGCCTGCTGTATGAACTGGCGCTGCCGGAAGAAGAAT | |
| | | GCCTGGAACGCCTGGAAGAAAACAACTATAACACCTAT | |
| | | AGCGATAAAAAATATCCGGATCCGGATTGGTATGTGGC | |
| | | GCTGAAAATTAAAACCGGCCAGTATGAACTGACCGGCC | |
| | | CGAAAACCGGCCCGCCGGATGGCCAGAAAGCGATTCT | |
| | | GTTTCTGCCGATGAGCGCGAAAAGC | |

1. A fibroblast growth factor (FGF) polypeptide comprising a sequence of SEQ ID NO: 4 or SEQ ID NO: 7, or a sequence with at least 80% sequence similarity to SEQ ID NO: 4 or SEQ ID NO: 7, wherein the polypeptide exhibits increased bioactivity, solubility and/or stability compared to the FGF2 polypeptide of SEQ ID NO: 1.

2. The FGF polypeptide of claim 1, wherein the polypeptide comprises a sequence of SEQ ID NO: 4 or SEQ ID NO: 7 or a sequence with at least 80% sequence similarity to SEQ ID NO: 4 or SEQ ID NO: 7, wherein the polypeptide binds a fibroblast growth factor receptor (FGFR) with greater affinity than the FGF2 polypeptide of SEQ ID NO: 1 binds a FGFR.

3. The FGF polypeptide of claim 1, wherein the polypeptide comprises a sequence of SEQ ID NO: 4 or a sequence with at least 90% or 95% sequence similarity to SEQ ID NO: 4.

4. The FGF polypeptide of claim 1, wherein the polypeptide comprises a sequence of SEQ ID NO: 7 or a sequence with at least 90% or 95% sequence similarity to SEQ ID NO: 7.

5. The FGF polypeptide of claim 1, wherein the polypeptide has a melting temperature of at least 55° C.

6. The FGF polypeptide of claim 1, wherein the polypeptide is soluble.

7. The FGF polypeptide ofclaim 1, wherein the polypeptide binds FGFR2 with greater affinity than the FGF2 polypeptide of SEQ ID NO: 1.

8. A nucleic acid comprising a sequence selected from the group consisting of:
(a) a nucleic acid sequence encoding an FGF polypeptide of claim 1;
(b) a nucleic acid sequence having at least 80% sequence similarity with the nucleic acid sequence of SEQ ID NO: 10 or SEQ ID NO: 13 and encoding an FGF polypeptide of claim 1;
(c) a nucleic acid sequence hybridizing with the complement of the nucleic acid sequence of SEQ ID NO: 10 or SEQ ID NO: 13 and encoding an FGF polypeptide of claim 1;
and
(d) a nucleic acid sequence which is the reverse complement of a nucleic acid sequence as defined in (a), (b) or (c).

9. The nucleic acid of claim 8 part (b) or part (c), wherein the nucleic acid sequence of SEQ ID NO: 10 or SEQ ID NO: 13 is the nucleic acid sequence of SEQ ID NO: 10.

10. The nucleic acid of claim 8 part (b) or part (c), wherein the nucleic acid sequence of SEQ ID NO: 10 or SEQ ID NO: 13 is the nucleic acid sequence of SEQ ID NO: 13.

11. A cell comprising the FGF polypeptide of claim 1 or a nucleic acid sequence encoding the FGF polypeptide.

12. The cell of claim 11, wherein the cell is a bacteria cell, a yeast cell, a plant cell, an insect cell, or a mammalian cell.

13. A method of preparing an FGF polypeptide, wherein the method comprises cultivating the cell of claim 11 under conditions which allow for expression of said polypeptide.

14. A method of preparing an FGF polypeptide, wherein the method comprises i) culturing a nucleic acid sequence of claim 8 in a cell lysate with ribosomal activity, and ii) synthesising a protein encoded by the nucleic acid with a cell-free protein synthesis reaction.

15. A method of growing an animal cell, wherein the method comprises cultivating the animal cell in a culture medium containing the FGF polypeptide of claim 1.

16. The method of claim 13, further comprising recovering the expressed polypeptide.


SEQUENCE LISTING (13 sequences)

SEQ ID NO: 1 - protein, 145 aa, Bos taurus
ALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYSSWYVALKRTGQYKLGPKTGPGQKAILFLPMSAKS

SEQ ID NO: 2 - protein, 147 aa, synthetic construct
AEIPEDGGSGAFPPGHFKDPKRLYGKNGGFFLRIEPDGRVEDAGVRESDPHIKLQLQAEERGVVLIKGVLANRYLAMKEDGRLLALKLPEEECLFLEREEENHYNTYRSDKYPDWYVALKRTGQYKLGPKTGPGQKAILFLPMSAKS

SEQ ID NO: 3 - protein, 144 aa, synthetic construct
ALPEDGGSGAFPPGHFKDPKLLVGKNGGFFLRIEDGKVDGVRESDPHIKLQLQAEERGVVLIKGVLANRYLAMKEDGRLLALKLPEEECFFERLEEENHYNTYRSDKKYPDWYVALKRTGQYKLGPKTGPGQKAILFLPMSAKS

SEQ ID NO: 4 - protein, 145 aa, synthetic construct
ALPEDGGSGAFPPGHFKDPKLLVDDGGFFLRIEPDGRVDGVREKSDPHIKLQLQAEERGVVLIKGVLANRYLAMKEDGRLLYASKLPEEECFFFERLEEENHYNTYSDKYPDWYVALKRTGQYKLGPKTGPGQKAILFLPMSAKS

SEQ ID NO: 5 - protein, 144 aa, synthetic construct
ALPEDGGSGAFPPGHFKDPKLLVDDGGFFLRIEPDGRVDGVREKSDPHIKLQLQAEERGVVLIKGVLANRYLAMKEDGRLFALPEATEECFFFERLEEENHYNTYSDKYPDWYVALKRTGQYKLGPKTGPGQKAILFLPMSAKS

SEQ ID NO: 6 - protein, 142 aa, synthetic construct
AEPEADGGAGAQPPGPDDGAEGGFFLRIEPPDGRVEVDGVRESDPHIKLQLQAEERGVVSIKGVLKYLAMKEDGRLLYELALPEEECLERLEENHYNTYISDKKYPDPDWYVALKIKGQYELTPKTGPGQKAILFLPMSAKS

SEQ ID NO: 7 - protein, 145 aa, synthetic construct
AEPEADGGAGAQPPGPDDGAEGFFLRIEPPDGRVEVDGVRESDPDKLQLQAEERGGVVTIKGVPDIYLAMKEDGRLLYELALPEEECLERLEENNYNTYSDKKYPDPDWYVALKIKTGQYELTGPKTGPPDGQKAILFLPMSAKS

SEQ ID NO: 8 - DNA, 441 bp, synthetic construct
  source 1..441: mol_type = other DNA
gcggaaattccggaagatggcggcagcggcgcgtttccgccgggccattttaaagatccgaaacgcctgtatggcaaaaacggcggcttttttctgcgcattgaaccggatggccgcgtggaagatgcgggcgtgcgcgaaagcgatccgcatattaaactgcagctgcaggcggaagaacgcggcgtggtgctgattaaaggcgtgctggcgaaccgctatctggcgatgaaagaagatggccgcctgctggcgctgaaactgccggaagaagaatgcctgtttctggaacgcgaagaagaaaaccattataacacctatcgcagcgataaatatccggattggtatgtggcgctgaaacgcaccggccagtataaactgggcccgaaaaccggcccgggccagaaagcgattctgtttctgccgatgagcgcgaaaagc

SEQ ID NO: 9 - DNA, 432 bp, synthetic construct
  source 1..432: mol_type = other DNA
gcgctgccggaagatggcggcagcggcgcgtttccgccgggccattttaaagatccgaaactgctggtgggcaaaaacggcggcttttttctgcgcattgaagatggcaaagtggatggcgtgcgcgaaagcgatccgcatattaaactgcagctgcaggcggaagaacgcggcgtggtgctgattaaaggcgtgctggcgaaccgctatctggcgatgaaagaagatggccgcctgctggcgctgaaactgccggaagaagaatgcttttttgaacgcctggaagaagaaaaccattataacacctatcgcagcgataaaaaatatccggattggtatgtggcgctgaaacgcaccggccagtataaactgggcccgaaaaccggcccgggccagaaagcgattctgtttctgccgatgagcgcgaaaagc

SEQ ID NO: 10 - DNA, 435 bp, synthetic construct
  source 1..435: mol_type = other DNA
gcgctgccggaagatggcggcagcggcgcgtttccgccgggccattttaaagatccgaaactgctggtggatgatggcggcttttttctgcgcattgaaccggatggccgcgtggatggcgtgcgcgaaaaaagcgatccgcatattaaactgcagctgcaggcggaagaacgcggcgtggtgctgattaaaggcgtgctggcgaaccgctatctggcgatgaaagaagatggccgcctgctgtatgcgagcaaactgccggaagaagaatgctttttttttgaacgcctggaagaagaaaaccattataacacctatagcgataaatatccggattggtatgtggcgctgaaacgcaccggccagtataaactgggcccgaaaaccggcccgggccagaaagcgattctgtttctgccgatgagcgcgaaaagc

SEQ ID NO: 11 - DNA, 432 bp, synthetic construct
  source 1..432: mol_type = other DNA
gcgctgccggaagatggcggcagcggcgcgtttccgccgggccattttaaagatccgaaactgctggtggatgatggcggcttttttctgcgcattgaaccggatggccgcgtggatggcgtgcgcgaaaaaagcgatccgcatattaaactgcagctgcaggcggaagaacgcggcgtggtgctgattaaaggcgtgctggcgaaccgctatctggcgatgaaagaagatggccgcctgtttgcgctgccggaagcgaccgaagaatgctttttttttgaacgcctggaagaagaaaaccattataacacctatagcgataaatatccggattggtatgtggcgctgaaacgcaccggccagtataaactgggcccgaaaaccggcccgggccagaaagcgattctgtttctgccgatgagcgcgaaaagc

SEQ ID NO: 12 - DNA, 426 bp, synthetic construct
  source 1..426: mol_type = other DNA
gcggaaccggaagcggatgggggcgcgggagcccaaccacctggtcctgacgatggtgctgagggcggtttttttctgcgcattgaaccgccggatggccgcgtggaagtggatggcgtgcgcgaaagcgatccgcatattaaactgcagctgcaggcggaagaacgcggcgtggtgagcattaaaggcgtgctgaaatatctggcgatgaaagaagatggccgcctgctgtatgaactggcgctgccggaagaagaatgcctggaacgcctggaagaaaaccattataacacctatattagcgataaaaaatatccggatccggattggtatgtggcgctgaaaattaaaggccagtatgaactgaccccgaaaaccggcccgggccagaaagcgattctgtttctgccgatgagcgcgaaaagc

SEQ ID NO: 13 - DNA, 435 bp, synthetic construct
  source 1..435: mol_type = other DNA
gcggaaccggaagcggatggcggcgcgggcgcgcagccgccgggcccggatgatggcgcggaaggcttttttctgcgcattgaaccgccggatggccgcgtggaagtggatggcgtgcgcgaaagcgatccggataaactgcagctgcaggcggaagaacgcggcggcgtggtgaccattaaaggcgtgccggatatttatctggcgatgaaagaagatggccgcctgctgtatgaactggcgctgccggaagaagaatgcctggaacgcctggaagaaaacaactataacacctatagcgataaaaaatatccggatccggattggtatgtggcgctgaaaattaaaaccggccagtatgaactgaccggcccgaaaaccggcccgccggatggccagaaagcgattctgtttctgccgatgagcgcgaaaagc